(19) United States
(12) Patent Application Publication
(10) Pub. No.: US 2023/0257698 A1
(43) Pub. Date: Aug. 17, 2023

(54) FETAL TROPHOBLAST COMPOSITIONS AND METHODS

(71) Applicant: NextGen Jane, Inc., Oakland, CA (US)

(72) Inventors: Stephen GIRE, Oakland, CA (US); Ridhi TARIYAL, Oakland, CA (US); Alain MIR, Oakland, CA (US)

(21) Appl. No.: 18/014,568

(22) PCT Filed: Jul. 6, 2021
(86) PCT No.: PCT/US2021/040503

§ 371 Date: Jan. 5, 2023

Related U.S. Application Data
(60) Provisional application No. 63/048,845, filed on Jul. 7, 2020.

(51) Int. Cl. C12N 5/073; A61B 10/02

(52) U.S. Cl. CPC C12N 5/0605; A61B 10/02; C12N 2500/60

(57) ABSTRACT

Provided herein are compositions comprising populations of cells obtained from a pregnant subject comprising at least one of a Hofbauer cell, a cytotrophoblast, a syncytiotrophoblast, an extravillous trophoblast, a villous core stroma, or a fetal vascular cell; a stabilization buffer, wherein the stabilization buffer comprises a preservative; and a cellulosic collection device, wherein at least a subset of the population of cells are intact and inviable and methods of making and using such compositions.

### CROSS-REFERENCE

This application claims the benefit of U.S. Patent Application No. 63/048,845, filed Jul. 7, 2020, which is hereby incorporated by reference in its entirety

### BACKGROUND

Fetal trophoblast cells, or fetal derived cells from the placenta are a source of information about the status of a pregnancy. In some cases, these cells are shed into vaginal secretions of a pregnant person and offer a less invasive route for collecting fetal derived cells as an alternative to chorionic villous sampling or amniocentesis.

### SUMMARY

In certain aspects, disclosed herein is a composition comprising: (a) a population of trophoblast and trophoblast-derived cells; (b) a stabilization buffer, wherein the stabilization buffer comprises a preservative; and (c) a cellulosic collection device, wherein at least a subset of the population of cells are intact and inviable. In some embodiments, the population of trophoblast and trophoblast-derived cells comprises one or more of a Hofbauer cell, a cytotrophoblast, a syncytiotrophoblast, an extravillous trophoblast, a villous core stroma, or a fetal vascular cell. In some embodiments, the cellulosic collection device comprises a tampon, a pad, a plug, or a swab. In some embodiments, at least a portion of the population of cells are associated with the cellulosic collection device. In some embodiments, at least 50%, at least 60%, or at least 70% of the population of cells are intact after 1, 2, 3, 4, 5, 6, 7, 8, 9, or 10 days in the stabilization buffer at room temperature. In some embodiments, the stabilization buffer maintains the size of the cells in the population within a range of 0.5 to 1× as compared with the size of the cells prior to contact with the stabilization buffer. In some embodiments, the population of cells comprises at least 100 cells or at least 500 cells.

In certain aspects, disclosed herein is a composition comprising: (a) a population of trophoblast and trophoblast-derived cells; and (b) a stabilization buffer, wherein the stabilization buffer comprises a preservative and wherein at least a subset of the population of cells are inviable and at least 50%, at least 60%, or at least 70% of the population of cells are intact after 1, 2, 3, 4, 5, 6, 7, 8, 9, or 10 days in the stabilization buffer at room temperature. In some embodiments, the population of trophoblast and trophoblast-derived cells comprises one or more of a Hofbauer cell, a cytotrophoblast, a syncytiotrophoblast, an extravillous trophoblast, a villous core stroma, or a fetal vascular cell. In some embodiments, the stabilization buffer maintains the size of the cells in the population within a range of 0.5 to 1× as compared with the size of the cells prior to contact with the stabilization buffer. In some embodiments, the stabilization buffer maintains the size of the cells in the population within a range of ±20%, ±10%, or ±5% as compared with the size of the cells prior to contact with the stabilization buffer. In some embodiments, the composition further comprises a cellulosic collection device.

In certain aspects, disclosed herein is a composition comprising: (a) an enriched population of intact and nonviable trophoblast and trophoblast-derived cells, wherein the population of cells comprises between 0.5% to 50% trophoblast and trophoblast-derived cells and at least 50%, at least 60%, or at least 70% of the enriched population are intact cells. In some embodiments, the population of cells comprises at least 0.5%, 1%, 2%, 5%, 10%, 20%, 25%, 30%, 40% or 50% trophoblast and trophoblast-derived cells. In some embodiments, the population of cells is enriched for trophoblast and trophoblast-derived cells between about 2-fold and 2000-fold. In some embodiments, the population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold. In some embodiments, the population of trophoblast and trophoblast-derived cells comprises one or more of a Hofbauer cell, a cytotrophoblast, a syncytiotrophoblast, an extravillous trophoblast, a villous core stroma, or a fetal vascular cell. In some embodiments, the enriched population of cells comprises at least two cell types selected from the group consisting of a cytotrophoblast, a syncytiotrophoblast, and extravillous trophoblast. In some embodiments, the enriched population of cells comprises at least cytotrophoblasts or syncytiotrophoblasts. In some embodiments, the composition further comprises a binding agent that specifically binds to a cell surface marker.

A method of obtaining a stabilized population of cells, the method comprising: (a) placing a cellulosic collection device in or near a vagina of a pregnant subject to collect vaginal fluid comprising a plurality of cells comprising trophoblast and trophoblast-derived cells; and (b) contacting the cellulosic collection device comprising the plurality of cells with a stabilization buffer, wherein at least a subset of the plurality of cells are intact and inviable. In some embodiments, the plurality of trophoblast and trophoblast-derived cells comprises one or more of a Hofbauer cell, a cytotrophoblast, a syncytiotrophoblast, an extravillous trophoblast, a villous core stroma, or a fetal vascular cell. In some embodiments, at least 50%, at least 60%, or at least 70% of the plurality of cells are intact after 1, 2, 3, 4, 5, 6, 7, 8, 9, or 10 days in the stabilization buffer at room temperature. In some embodiments, the stabilization buffer maintains the size of the cells in the plurality within a range of 0.5 to 1× as compared with the size of the cells prior to contact with the stabilization buffer. In some embodiments, the stabilization buffer maintains the size of the cells in the plurality within a range of ±20%, ±10%, or ±5% as compared with the size of the cells prior to contact with the stabilization buffer. In some embodiments, the plurality of cells comprises at least two of a Hofbauer cell, a cytotrophoblast, a syncytiotrophoblast, an extravillous trophoblast, a villous core stroma, or a fetal vascular cell. In some embodiments, the plurality of cells comprises at least one of a cytotrophoblast or a syncytiotrophoblast. In some embodiments, the cellulosic collection device is in or near the vagina for at least 10, 20, 30, 40, 50, 60, 90, 120, 150, 180, 240, 300, 360, 420, or 480 minutes. In some embodiments, the method further comprises eluting the plurality of cells or a portion thereof from the cellulosic collection device to obtain an isolated stabilized population of cells. In some embodiments, the method further comprises subjecting the isolated stabilized population of cells to one or more enrichment steps to obtain an enriched population of cells. In some embodiments, the enriched population of cells is enriched in trophoblast cells, trophoblast-derived cells, or trophoblast and trophoblast-derived cells. In some embodiments, the enriched population of cells is enriched in one or more of Hofbauer cells, cytotrophoblasts, syncytiotrophoblasts, extravillous trophoblasts, villous core stroma cells, or fetal vascular cells. In some embodiments, the enriched population of cells comprises at least 0.5%, 1%, 2%, 5%, 10%, 20%, 25%, 30%, 40% or 50% trophoblast and trophoblast-derived cells. In some embodiments, the enriched population of cells is enriched for trophoblast and trophoblast-derived cells between about 2-fold and 2000-fold. In some embodiments, the enriched population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold. In some embodiments, the enriched population of cells is depleted of maternal cells. In some embodiments, the enriched population of cells is depleted of maternal cells between about 80% to about 99.9% as compared to the isolated stabilized population of cells. In some embodiments, the enriched population of cells comprises between about 50% to about 99.5% maternal cells. In some embodiments, the enrichment step comprises contacting with a binding agent that specifically binds to a cell surface marker, flow cytometry, density gradient centrifugation, and nuclei isolation. In some embodiments, the method comprises 2, 3 or 4 enrichment steps. In some embodiments, the enrichment step comprises contacting with a binding agent that specifically binds to a cell surface marker and separating at least a subset of the cells bound by the one or more binding agents to thereby form the enriched population of cells. In some embodiments, the cell surface marker is a fetal cell surface marker. In some embodiments, the cell surface marker is a maternal cell surface marker. In some embodiments, the cell surface marker comprises FSHR, CD45, LHCGR, CD270, Trop2, FOLR1, CD114 (CSF3R) or SLC40A1. In some embodiments, the binding agent is selected from the group consisting of an antibody, antibody fragment, a magnetic bead and a detectable label. In some embodiments, the cell surface marker is a maternal cell surface marker and the enriched population is depleted of maternal cells. In some embodiments, the cell surface marker is a fetal cell surface marker and the enriched population is enriched in placenta derived fetal cells. In some embodiments, the cell surface marker is a fetal cell surface marker and the enriched population is enriched in fetal trophoblast cells. In some embodiments, the method comprises quantifying a fetal fraction of the plurality of cells. In some embodiments, the method comprises quantifying a fetal fraction comprises intracellular staining, RNA analysis, short tandem repeat (STR) analysis, single nucleotide polymorphism (SNP) analysis, Y-chromosome analysis, or combinations thereof. In some embodiments, the cellulosic collection device is a tampon, a pad, a plug, or a swab. In some embodiments, the method further comprises removing mucus from the plurality of cells. In some embodiments, the method further comprises analyzing at least one of a nucleic acid, a protein, a carbohydrate, or a lipid from the enriched population of cells. In some embodiments, the method further comprises recommending a treatment to the pregnant subject. In some embodiments, the method further comprises analyzing at least one of a nucleic acid, wherein the feature comprises a copy number variant, a single nucleotide variant, an insertion, a deletion, an epigenetic modification, a post-translational protein modification, or a combination thereof. In some embodiments, the epigenetic modification comprises methylation, phosphorylation, ubiquitination, sumoylation, acetylation, ribosylation, citrullination, or a combination thereof. In some embodiments, the method further comprises labeling at least one of the nucleic acids prior to analysis. In some embodiments, the analysis comprises one or more of PCR, sequencing, karyotyping, in situ hybridization, immunofluorescence, FACS, or mass spectroscopy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 illustrates a bar graph of antibody labeling fetal and maternal cells.

FIG. 2 illustrates a bar graph of change in the number of cells by day in each buffer.

FIG. 3 illustrates a bar graph of change in the number of cells by day in a selection of buffers.

FIG. 4 illustrates a bar graph of fetal enrichment of trophoblast cells spiked into cervicovaginal samples.

FIG. 5 illustrates a bar graph of the percent of cell shearing at different centrifuge speeds.

FIG. 6 depicts a forward scatter pattern for an exemplary sample analyzed using flow cytometry.

FIG. 7 depicts flow cytometry data for samples taken from pregnant and non-pregnant controls.

FIG. 8 depicts the ratios o dead and live cells for samples preserved for 0 to 5 days.

FIG. 9 depicts exemplary cell layers prepared using discontinuous density gradients.

FIG. 10 depicts cell samples at 10× and 20× magnification collected from different density layers.

FIG. 11 depicts the fetal fraction of each cell density layer.

FIG. 12 depicts an analysis of the abundance of different cell types identified in the cervicovaginal samples.

FIG. 13 depicts reduction in autofluorescence of one of the 5 samples analyzed.

FIG. 14 depicts the fold change enrichment in percent of fetal fraction for each antibody and blocking reagent tested.

### DETAILED DESCRIPTION

Monitoring the health of a pregnancy is an important aspect of prenatal care. The advent of non-invasive pre-natal testing (NIPT) allowed for testing the chromosomal content of a fetus based on cell-free nucleic acids in the blood of a pregnant person. However, this can give an incomplete picture of the fetal genetics and obtaining fetal cells via chorionic villus sampling or amniocentesis is invasive and has a small risk of miscarriage. Therefore, it is desired to obtain fetal derived cells, such as trophoblasts and other placental derived fetal cells in a non-invasive manner. Provided herein are methods of obtaining fetal trophoblast cells and other placental derived cells from the vaginal fluids of a pregnant person using a cellulosic collection device, and compositions obtained via these methods. These methods result in compositions having inviable, intact cells that can be used to monitor the health of a pregnancy.

Provided herein are compositions comprising fetal trophoblast cells or other placental derived cells (referred to herein collectively as “trophoblast-derived cell populations” and “cells derived from the trophectoderm”) derived from vaginal secretions of pregnant subjects and methods of collecting and analyzing these cells. In some cases, collection of these cells allows for a non-invasive prenatal testing via collection of fetal trophoblast cells in vaginal fluid using a cellulosic collection device, such as a tampon. In methods of collection described herein, intact, inviable cells are obtained by the cellulosic collection device and transported for analysis in a stabilization buffer that maintains the integrity of the cells during transport.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number, in some cases, is a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein. In some cases, the upper and lower limits of these smaller ranges are independently included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

As used herein, the terms “subject,” “individual,” and “patient” are used interchangeably. None of the terms are to be interpreted as requiring the supervision of a medical professional (e.g., a doctor, nurse, physician's assistant, orderly, or hospice worker). In some cases, the subject is any animal, including mammals (e.g., a human or non-human animal). In one embodiment of the methods and compositions provided herein, the mammal is a human.

The term “nucleic acid,” as used herein, generally refers to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), guide RNA (gRNA), messenger RNA (mRNA), cell-free DNA (cfDNA), cell-free RNA (cfRNA), micro RNA (miRNA), DNA-RNA hybrids, or a polymer comprising purine and pyrimidine bases or other natural, chemically or biochemically modified, non-natural, or derivatized nucleotide bases.

The term “stabilization buffer,” as used herein, refers to an aqueous buffer that maintains a substantial proportion of cells in the stabilization buffer as intact, inviable (or nonproliferating) and substantially not expanding or contracting in size (e.g., +/−25%). In some embodiments, stabilization buffers herein comprise a preservative.

The term “preservative,” as used herein, refers to an agent added to a composition, such as a composition comprising cells, in order to prevent breakdown of biological components of the composition (e.g., cells). In some embodiments, preservatives herein maintain a composition comprising cells, keeping the cells inviable and intact without refrigeration. In embodiments herein, a preservative maintains the size of cells in the composition (i.e., does not cause the cells to shrink or expand). In some embodiments, a preservative may comprise a chelating agent (e.g., EDTA), a polyol (e.g., glycerol), a zwitterion (e.g., glycerol phosphate or pentaerythritol), an osomprotectants/pH buffer (e.g., Ca⁺, Cl⁻, K⁺, tartaric acid), or combinations thereof.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Fetal Trophoblast and Placental Cell Compositions

Provided herein are compositions comprising trophoblast-derived cell populations. In some embodiments, the composition comprises at least one of a Hofbauer cell, a cytotrophoblast, a syncytiotrophoblast, an extravillous trophoblast, a villous core stroma, or a fetal vascular cell. In some embodiments, the composition comprises a cytotrophoblast, a syncytiotrophoblast, and an extravillous trophoblast. In some embodiments, the composition comprises an extravillous trophoblast and a cytotrophoblast. In some embodiments, the composition comprises an extravillous trophoblast and a syncytiotrophoblast. In some embodiments, the composition further comprises a stabilization buffer, wherein the stabilization buffer comprises a preservative. In some embodiments, the composition further comprises a cellulosic collection device, wherein at least a subset of the population of cells are intact and inviable.

In additional aspects, there are provided compositions comprising a population of cells comprising trophoblast and trophoblast-derived cells, such as at least one of a Hofbauer cell, a cytotrophoblast, a syncytiotrophoblast, an extravillous trophoblast, a villous core stroma, or a fetal vascular cell and a stabilization buffer, wherein the stabilization buffer comprises a preservative and wherein at least a subset of the population of cells are intact and inviable.

In further aspects, there are provided compositions comprising an enriched population of intact and nonviable trophoblast and trophoblast-derived cells, wherein the proportion of maternal cells in the population is between about 50% to 99.5% of the enriched population, and where the trophoblast and trophoblast-derived cells comprise between 0.5% and 50% of the enriched population; and at least 50%, at least 60%, or at least 70% of the enriched population are intact cells. In some embodiments, from about 20% to about 70%, from about 20% to about 60%, from about 20% to about 50%, from about 30% to about 70%, from about 30% to about 60%, from about 30% to about 50%, from about 40% to about 70%, from about 40% to about 60%, from about 40% to about 50%, from about 50% to about 70%, from about 50% to about 60%, or from about 60% to about 70% of the enriched population of cells are intact cells. In some embodiments the proportion of maternal cells in the population is less than about 50% or less than about 20%.

In further aspects, there are provided compositions comprising an enriched population of intact and nonviable trophoblast and trophoblast-derived cells, wherein the proportion of maternal cells in the population has been depleted, In some embodiments, maternal cells are depleted 10 fold, 20 fold, 50 fold, 100 fold or more than 100 fold. In some embodiments, maternal cells are depleted from the starting population of cells at least 80% depletion, 85% depletion, 90% depletion, 95% depletion, 98% depletion or 99% depletion. In further aspects, there are provided compositions comprising an enriched population of intact and nonviable trophoblast and trophoblast-derived cells, wherein the proportion of trophoblast and trophoblast-derived cells are enriched as compared to the starting population, In some embodiments, the enrichment is at least 10 fold, 20 fold, 25 fold, 50 fold, 100 fold, 200 fold, 250 fold, 400 fold, 500 fold, 800 fold, 1000 fold, 1400 fold, 1500 fold, 2000 fold or more than 2000 fold.

In some aspects of compositions herein, compositions comprise a cellulosic collection device. In some cases, the cellulosic collection device comprises any suitable device which rests near or inside a vagina for the purposes of collecting vaginal fluid or secretions. In some cases, the cellulosic collection device comprises a tampon, a pad, a plug, or a swab. In some cases, at least a portion of the population of cells are associated with the cellulosic collection device.

In additional aspects, there are provided compositions comprising a population of cells comprising trophoblast and trophoblast-derived cells, such as at least one of a Hofbauer cell, a cytotrophoblast, a syncytiotrophoblast, an extravillous trophoblast, a villous core stroma, or a fetal vascular cell; and one or more binding agents, wherein the one or more binding agents is capable of specifically binding to a cell surface protein.

In some embodiments, compositions herein comprise cells wherein at least 50% of the population of cells is intact. For example, in some embodiments, at least 55%, at least 60%, at least 65%, at least 70%, at least 75%, at least 80%, at least 85%, at least 90%, at least 95%, or more of the population of cells are intact. In some embodiments, at least 50% of the population of cells is intact after at least 1 day in the stabilization buffer. For example, in some embodiments, at least 50% of the population of cells is intact after at least 2 days, at least 3 days, at least 4 days, at least 5 days, at least 6 days, at least 7 days, at least 8 days, at least 9 days, at least 10 days, at least 11 days, at least 12 days, at least 13, days, at least 14 days, or more in the stabilization buffer. In some cases, at least 50% of the population of cells is intact after at least 1 day in the stabilization buffer at room temperature. In some cases, room temperature is about 10° C., about 15° C., about 20° C., about 25° C., about 30° C., or about 35° C. In some cases, room temperature is from about 10° C. to about 35° C., from about 10° C. to about 30° C., from about 10° C. to about 25° C., from about 10° C. to about 20° C., from about 10° C. to about 15° C., from about 15° C. to about 35° C., from about 15° C. to about 30° C., from about 15° C. to about 25° C., from about 15° C. to about 20° C., from about 20° C. to about 35° C., from about 20° C. to about 30° C., from about 20° C. to about 25° C., from about 25° C. to about 35° C., from about 25° C. to about 30° C., or from about 30° C. to about 35° C.

In some embodiments, compositions herein comprise cells wherein the stabilization buffer maintains the size of the cells in the population as compared with the size of the cells prior to contact with the stabilization buffer. In some embodiments, the cells are maintained at a size at least 0.5× as compared with the size of the cells prior to contact with the stabilization buffer. For example, in some embodiments, cells are maintained at a size at least 0.6,×, at least 0.7×, at least 0.8×, at least 0.9×, or at least 1× as compared with the size of the cells prior to contact with the stabilization buffer. In some embodiments, cells are maintained at a size within a range of 0.5 to 1×, 0.5 to 0.9×, 0.5 to 0.8×, 0.5 to 0.7×, 0.5 to 0.6×, 0.6 to 1×, 0.6 to 0.9×, 0.6 to 0.8×, 0.6 to 0.7×, 0.7 to 1×, 0.7 to 0.9×, 0.7 to 0.8×, 0.8 to 1×, 0.8 to 0.9×, or 0.9 to 1× as compared with the size of the cells prior to contact with the stabilization buffer. In some embodiments, the stabilization buffer maintains the size of the cells in the population within a range of ±20% as compared with the size of the cells prior to contact with the stabilization buffer. In some embodiments, the stabilization buffer maintains the size of the cells in the population within a range of ±15% as compared with the size of the cells prior to contact with the stabilization buffer. In some embodiments, the stabilization buffer maintains the size of the cells in the population within a range of ±10% as compared with the size of the cells prior to contact with the stabilization buffer. In some embodiments, the stabilization buffer maintains the size of the cells in the population within a range of ±5% as compared with the size of the cells prior to contact with the stabilization buffer.

In some embodiments, compositions herein comprise a enriched population of cells comprising trophoblast and trophoblast-derived cells, such as Hofbauer cells, cytotrophoblasts, syncytiotrophoblasts, extravillous trophoblasts, villous core stromas, or fetal vascular cells. In some embodiments, compositions comprise an enriched population of cells comprising at least two of Hofbauer cells, cytotrophoblasts, syncytiotrophoblasts, extravillous trophoblasts, villous core stromas, or fetal vascular cells. In some embodiments, compositions comprise an enriched population of cells comprising at least three of Hofbauer cells, cytotrophoblasts, syncytiotrophoblasts, extravillous trophoblasts, villous core stromas, or fetal vascular cells. In some embodiments, compositions comprise an enriched population of cells comprising at least four of Hofbauer cells, cytotrophoblasts, syncytiotrophoblasts, extravillous trophoblasts, villous core stromas, or fetal vascular cells. In some embodiments, compositions comprise an enriched population of cells comprising at least five of Hofbauer cells, cytotrophoblasts, syncytiotrophoblasts, extravillous trophoblasts, villous core stromas, or fetal vascular cells. In some embodiments, the enriched population of cells comprises at least one of cytotrophoblasts or syncytiotrophoblasts. In some embodiments, the enriched population of cells comprises cytotrophoblasts and syncytiotrophoblasts.

In some embodiments, compositions comprising a population of cells provided herein further comprise a binding agent that specifically binds to a cell surface marker. In some embodiments, the cell surface marker is a fetal cell surface marker. In some embodiments, the cell surface marker is a maternal cell surface marker. In some embodiments, the cell surface marker comprises FSHR, CD45, LHCGR, CD270, Trop2, FOLR1, CD114 (CSF3R) or SLC40A1. In some embodiments, the cell surface marker comprises FSHR, CD45, LHCGR, CD270, CD156a, or ERVW1. In some embodiments, the cell surface marker comprises Trop2, GPC3, TREML2, ALPP (866), CD163, CD144, ALPP (GM022), FOLR1, CD114 (CSF3R), CD115, TSPAN1, SLC22A1, EFNA1, SLC62A2, SLC40A1, CD71, or CD166. In some embodiments, the composition comprises extravillous trophoblasts and the cell surface marker comprises at least one of TGF-beta2, human placental lactogen, c-erbB2, PAPPA2, or PRG2. In some embodiments, the composition comprises syncytiotrophoblasts and the cell surface marker comprises at least one of cytokeratin 7, beta-HCG, alpha-HCG, GCM1, syncytin, c-erbB2, leptin, INSL4, TGF-beta1, CSH1, or KISS1. In some embodiments, the composition comprises cytotrophoblsts and the cell surface marker comprises at least one of INSL4, PEG10, PAGE4, or p63.

In some embodiments of compositions herein, the binding agent is an antibody or fragment thereof. In some embodiments, the binding agent comprises an IgG, IgD, IgM, IgA, or IgE. In some embodiments, the binding agent comprises an IgG1, and IgG2, IgG3 or IGg4. In some embodiments, the binding agent is a monoclonal antibody. In some embodiments, the binding agent is a polyclonal antibody. In some embodiments, the binding agent is an antigen binding fragment. In some embodiments, the binding agent is a Fab fragment, F(ab′)2 fragment, single chain Fv (scFv), diabody, triabody, or minibody. In some embodiments, the binding agent comprises a bead, such as a magnetic bead. In some embodiments, the binding agent comprises biotin or streptavidin. In some embodiments, the binding agent comprises a detectable label. In some embodiments, the detectable label comprises a fluorophore, such as a Fluorescein (FITC), an Allophycocyanin (APC), a R-Phycoerythrin (PE), a green fluorescent protein (GFP), a red fluorescent protein (RFP), a yellow fluorescent protein (YFP), or combination thereof.

In some embodiments of compositions herein, the population of cells comprises at least 500 cells. In some embodiments, the population of cells comprises at least 500 cells, at least 600 cells, at least 700 cells, at least 800 cells, at least 900 cells, at least 1000 cells, at least 1200 cells, at least 1500 cells, at least 1800 cells, at least 2000 cells, or more. In some embodiments, the population of cells comprises from about 500 cells to about 20000 cells, from about 500 cells to about 10000 cells, from about 500 cells to about 8000 cells, from about 500 cells to about 7500 cells, from about 500 cells to about 5000 cells, from about 500 cells to about 2500 cells, from about 500 cells to about 2000 cells, from about 500 cells to about 1800 cells, from about 500 cells to about 1500 cells, from about 500 cells to about 1200 cells, from about 500 cells to about 1000 cells, from about 500 cells to about 900 cells, from about 500 cells to about 800 cells, from about 500 cells to about 700 cells, or from about 500 cells to about 600 cells.

In some embodiments of compositions herein, the population of enriched cells comprises between about 0.5% to 50% trophoblasts and trophoblast-derived cells. In some embodiments, the population of enriched cells comprises at least about 0.5%, at least about 1%, at least about 5%, at least about 10%, at least about 20%, at least about 30%, at least about 40%, or more trophoblasts and trophoblast-derived cells. In some embodiments, the population of enriched cells comprises at least about 0.5% to about 2% trophoblast and trophoblast-derived cells, at least about 1% to about 50% trophoblasts and trophoblast-derived cells, at least about 5% to about 10% trophoblasts and trophoblast-derived cells, at least about 10% to about 20% trophoblasts and trophoblast-derived cells, at least about 20% to about 30% trophoblasts and trophoblast-derived cells, at least about 30% to about 40% trophoblasts and trophoblast-derived cells, or at least about 40% to about 50% trophoblasts and trophoblast-derived cells. In some embodiments, the population of enriched cells comprises about 0.5% to 1%, 1% to 2%, 2% to 4%, 3% to 5%, 4% to 6%, 5% to 7%, 5% to 10%, 10% to 15%, 15% to 20%, 15% to 25%, 20% to 40%, 20% to 50%, 30% to 50%, 40% to 50% trophoblasts and trophoblast-derived cells. In some embodiments, the population of enriched cells comprises between about 50% to 99.5% maternal cells. For example, in some embodiments, the population of enriched cells comprises no more than about 99.5%, about 99%, about 90%, about 80% maternal cells, about 70% maternal cells, about 60% maternal cells, or about 50% maternal cells. In some embodiments, the population of enriched cells comprises no more than about 99-99.5%, 95-99%, 90-95%, 80-90%, 70-80%, 60-70%, or 50-60% maternal cells.

In some embodiments of compositions herein, the population of enriched cells comprises at least 50% trophoblasts and trophoblast-derived cells. In some embodiments, the population of enriched cells comprises at least about 50%, at least about 60%, at least about 70%, at least about 80%, or more trophoblasts and trophoblast-derived cells. In some embodiments, the population of enriched cells comprises at least about 40% to about 50% trophoblast, at least about 50% to about 60% trophoblasts and trophoblast-derived cells, at least about 60% to about 70% trophoblasts and trophoblast-derived cells, at least about 70% to about 80% trophoblasts and trophoblast-derived cells, at least about 50% to about 70% trophoblasts and trophoblast-derived cells, at least about 50% to about 80% trophoblasts and trophoblast-derived cells, or at least about 60% to about 80% trophoblasts and trophoblast-derived cells. In some embodiments, the population of enriched cells comprises about 50% to 60%, 50% to 70%, 50% to 80%, 50% to 90%, 50% to 95%, 50% to 99%, 60% to 70%, 60% to 80%, 60% to 90%, 60% to 95%, 60% to 99%, 70% to 80%, 70% to 90%, 70% to 95%, 70% to 99%, 80% to 90%, 80% to 95%, 80% to 99%, 90% to 95%, 90% to 99%, or 95% to 99% trophoblasts and trophoblast-derived cells. In some embodiments, the population of enriched cells comprises no more than about 50% maternal cells. For example, in some embodiments, the population of enriched cells comprises no more than about 50%, about 40%, about 30%, about 20% maternal cells, about 10% maternal cells, about 5% maternal cells, or about 1% maternal cells. In some embodiments, the population of enriched cells comprises no more than about 1% to 5%, 1% to 10%, 1% to 20%, 1% to 30%, 1% to 40%, 1% to 50%, 5% to 10%, 5% to 20%, 5% to 30%, 5% to 40%, 5% to 50%, 10% to 20%, 10% to 30%, 10% to 40%, 10% to 50%, 20% to 30%, 20% to 40%, 20% to 50%, 30% to 40%, 30% to 50%, or 30% to 50% maternal cells.

In some embodiments, a portion of the population of cells that comprises trophoblasts comprises extravillous trophoblasts. In some embodiments, a portion of the population of cells that comprises trophoblasts comprises cytotrophoblasts. In some embodiments, a portion of the population of cells that comprises trophoblasts comprises syncytiotrophoblasts. In some embodiments, a portion of the population of cells that comprises trophoblasts comprises extravillous trophoblasts, cytotrophoblasts, and syncytiotrophoblasts. In some embodiments, a trophoblast composition has a fetal trophoblast to background ratio of 1/400 prior to enrichment.

In some embodiments of compositions herein, the composition comprises a stabilization buffer. In some embodiments, the stabilization buffer comprises at least one of: a preservation agent, a dissociation agent, or a combination thereof. In some embodiments, the stabilization buffer comprises a zwitterionic compound, an osmoprotectant, an apoptosis inhibitor, a non-reducing sugar or polyol, a disaccharide derivative, a chelating agent, a pH buffer, a phosphatase inhibitor, a protease inhibitor, or a combination thereof. In some embodiments, the dissociation agent comprises a mucolytic, an expectorant, a surfactant, a nuclease, a protease, or a combination thereof. In some embodiments, the stabilization buffer comprises a zwitterionic compound, an osmoprotectant, an apoptosis inhibitor, a non-reducing sugar or polyol, a chelating agent, a pH buffer, a phosphatase inhibitor, a protease inhibitor, a mucolytic, an expectorant, a surfactant, a nuclease, a protease, or any combination thereof. In some embodiments, the stabilization buffer comprises phosphate buffered saline, ethylenediaminetetraacetic acid, fetal bovine serum, Hank's balanced salt solution, collagenase, accutase, a nuclease, or combinations thereof.

In some embodiments, the stabilization buffer comprises an osmoprotectant. In some embodiments, the osmoprotectant comprises trimethylammonium acetate; glycerol phosphate; diglycerol phosphate, N-(2-hydroxy-1,1-bis(hydroxymethyl)ethyl)glycine; 3-(N-morpholino)-2-hydroxypropanesulfonic acid; pentaerythritol; glyceric acid; malic acid; tartaric acid; lactic acid; glycolic acid; 2-hydroxybutyric acid; 3-hydroxybutyric acid; 4-amino-3-hydroxybutyric acid; 3-(1-azoniabicyclo[2.2.2]oct-1-yl)propane-1-sulfonate; 1-(2-carboxylatoethyl)-1-azabicyclo[2.2.2]octan-1-ium; or any combination thereof.

In some embodiments, the stabilization buffer comprises an apoptosis inhibitor. In some embodiments, the apoptosis inhibitor comprises PERK-eIF2-α inhibitor, ASK1 inhibitor, NRF2-KEAP1 inhibitor, JNK inhibitor, p38 MAP kinase inhibitor, IRE1 inhibitor, GSK3 inhibitor, PIK3 pathway inhibitor, MEK inhibitor, calpain inhibitor, caspase-1 inhibitor, or any combination thereof.

In some embodiments, the stabilization buffer comprises a non-reducing sugar or polyol. In some embodiments, the non-reducing sugar or polyol comprises glycol, glycerol, erythritol, threitol, arabitol, xylitol, ribitol, adonitol, mannitol, sorbitol, galactitol, fucitol, iditol, inositol, adonitol, sucralfate, sucrose octasulfate, sucrose, trehalose, or any combination thereof. In some embodiments, the stabilization buffer comprises a disaccharide derivative. In some embodiments, the disaccharide derivative comprises sucralose, trichloronated maltose, or a combination thereof.

In some embodiments, the stabilization buffer comprises a chelating agent. In some embodiments, the chelating agent comprises diethylenetriaminepentaacetic acid (DTPA); ethylenediaminetetraacetic acid (EDTA); ethylene glycol tetraacetic acid (EGTA); trans-1,2-diaminocyclohexane-N,N,N′,N′-tetraacetic acid (CDTA); 1,2-bis(2-aminophenoxy)ethane-N,N,N′,N′-tetraacetic acid (BAPTA); 1,4,7,10-tetraazacyclododecane-1,4,7,10-tetraacetic acid (DOTA); N-(2-hydroxyethyl)ethylenediamine-N,N′,N′-triacetic acid; sodium gluconate; nitrilotriacetic acid (NTA); or a combination thereof.

In some embodiments, the stabilization buffer comprises a buffer. In some embodiments, the buffer comprises citric acid; tartaric acid; malic acid; sulfosalicylic acid; sulfoisophthalic acid; oxalic acid; borate; CAPS (3-(cyclohexylamino)-1-propanesulfonic acid); CAPSO (3-(cyclohexylamino)-2-hydroxy-1-propanesulfonic acid); EPPS (4-(2-hydroxyethyl)-1-piperazinepropanesulfonic acid); HEPES (4-(2-hydroxyethyl)piperazine-1-ethanesulfonic acid); MES (2-(N-morpholino)ethanesulfonic acid); MOPS (3-(N-morpholino)propanesulfonic acid); MOPSO (3-morpholino-2-hydroxypropanesulfonic acid); PIPES (1,4-piperazinediethanesulfonic acid); TAPS (N[tris(hydroxymethyl)methyl]-3-aminopropanesulfonic acid); TAPSO (2-hydroxy-3-[tris(hydroxymethyl)methylamino]-1-propanesulfonic acid); TES (N-[tris(hydroxymethyl)methyl]-2-aminoethanesulfonic acid); bicine (N,N-bis(2-hydroxyethyl)glycine); tricine (N-[tris(hydroxymethyl)methyl]glycine); tris (tris(hydroxymethyl)aminomethane); bis-tris (2-[bis(2-hydroxyethyl)amino]-2-(hydroxymethyl)-1,3-propanediol); or a combination thereof.

In some embodiments, the stabilization buffer comprises a phosphatase inhibitor. In some embodiments, the phosphatase inhibitor comprises beta-Glycerophosphate, aprotinin, bestatin, EDTA, leupeptin, pepstatin A, or a combination thereof.

In some embodiments, the stabilization buffer comprises a protease inhibitor. In some embodiments, the protease inhibitor comprises (2R)-2-Mercaptomethyl-4-methylpentanoyl-beta-(2-naphthyl)-Ala-Ala Amide; 2-Antiplasmin; 3,4-Dichloroisocoumarin; 4-(2-Aminoethyl) benzenesulfonyl fluoride hydrochloride; 5-(R,S)-T-trans-Cinnamido-7-methyl-4-oxo-octanoyl-L-prolyl-L-proline; a1-Antchymotrypsin; a1-Antitrypsin; a2-Antiplasmin; a2-Macroglobulin; Antithrombin III; Aprotinin; Bromoenol lactone; BTEE; C1 Esterase inhibitor; Chymostatin; Complement C1 esterase inhibitor; Dichloromethylenediphosphonic acid disodium salt; Diisopropyl fluorophosphate; e-Amino-n-caproic acid; Ecotin; EDTA; Eglin C fragment 60-63 methyl ester; Gabexate mesylate; Histatin 5; Ile-Pro-Ile; Isoamylphosphonyl-Gly-L-Pro-L-Ala; Leupeptin; N a-p-Tosyl-L-lysine chloromethyl ketone hydrochloride; N-Acetyl-eglin C; N-Tosyl-L-phenylalanine chloromethyl ketone; p-Chloromercuribenzoic acid Free Acid; Phenylmethylsulfonyl fluoride; Trypsin Inhibitor; Trypsin-chymotrypsin inhibitor; Z-L-Phe chloromethyl ketone; Boc-Asp(OMe)-fluoromethyl ketone; Z-Ala-Glu(OMe)-Val-Asp(OMe)-fluoromethyl ketone; Antipain dihydrochloride from microbial source protease inhibitor; CA-074 methyl ester; Calpain Inhibitor I; Calpain Inhibitor II; Cystatin; E-64 protease inhibitor; Leupeptin trifluoroacetate salt; α2-Macroglobulin; Procathepsin B; Z-Leu-Leu-Leu-fluoromethyl ketone; Z-Phe-Phe-fluoromethyl ketone; or a combination thereof.

In some embodiments, the stabilization buffer comprises a mucolytic agent. In some embodiments, the mucolytic agent dissociates (e.g., “unclump”) at least a portion of cellular aggregations in the cervicovaginal sample. In some embodiments, the mucolytic comprises acetylcysteine, ambroxol, bromhexine, carbocisteine, domiodol, domase alfa, eprazinone, erdosteine, letosteine, mannitol, mesna, neltenexine, sobrerol, stepronin, tiopronin, N-acetyl-L-cysteine, L-acetyl cysteine/Liberase™, or a combination thereof.

In some embodiments, the stabilization buffer comprises an expectorant. In some embodiments, the expectorant comprises althea root, antimony pentasulfide, creosote, guaiacolsulfonate, guaifenesin (+oxomemazine), ipecacuanha, levoverbenone, potassium iodide, senega, tyloxapol, ammonium chloride, or a combination thereof.

In some embodiments, the stabilization buffer comprises a surfactant. In some embodiments, the surfactant comprises polyoxyethylene glycol octylphenol ethers; polyoxyethylene glycol alkylphenol ethers; polyoxyethylene glycol sorbitan alkyl esters; sorbitan alkyl esters; polyethylene glycol; polypropylene glycol; carboxylates; sulphonates; petroleum sulphonates; alkylbenzenesulphonates; naphthalenesulphonates; olefin sulphonates; alkyl sulphates; sulphates; sulphated esters; sulphated alkanolamides; alkylphenols; ethoxylated aliphatic alcohol; polyoxyethylene surfactants; carboxylic esters; polyethylene glycol esters; anhydrosorbitol esters; glycol esters; carboxylic amide; monoalkanolamine condensates; polyoxyethylene fatty acid amides; quaternary ammonium salts; polyoxyethylene alkyl and alicyclic amines; N,N,N′,N′ tetrakis substituted ethylenediamines; 2-alkyl 1-hydroxyethyl 2-imidazolines; or a combination thereof.

In some embodiments, the stabilization buffer comprises a nuclease. In some embodiments, the nuclease comprises Benzonase®, DNAse I, DNAse II, Exonuclease III, Micrococcal Nuclease, Nuclease P1, Nuclease S1, Phosphodiesterase I, Phosphodiesterase II, RNAse A, RNAse H, RNAse T1, or a combination thereof.

In some embodiments, the stabilization buffer comprises a protease. In some embodiments, the protease comprises adispase II, trypsin, pronase, collagenase 1, collagenase 2, collagenase 3, collagenase 4, hyaluronidase, pepsin, papain, chemotrypsin, chymase, clostripain, complement C1r, complement C1s, complement factor D, complement factor I, cucumisin, dipeptidyl peptidase, elastase, endoproteinase, enterokinase, Factor X Activated, caspase, cathepsin, matrix metalloprotease, or a combination thereof.

In some embodiments, the osmolality of the preservation solution is from about 310 to about 410 mOsm kg-1. In some embodiments, the osmolality of the preservation solution is from about 95 to about 210 mOsm kg-1.

### Methods of Collection and Analysis

In additional aspects, there are provided methods of obtaining a stabilized population of cells. In some embodiments the method comprises placing a cellulosic collection device in or near a vagina of a pregnant subject to collect vaginal fluid comprising a plurality of cells comprising trophoblast and trophoblast-derived cells, such as comprising at least one of a Hofbauer cell, a cytotrophoblast, a syncytiotrophoblast, an extravillous trophoblast, a villous core stroma, or a fetal vascular cell. In some embodiments, the method comprises contacting the cellulosic collection device comprising the plurality of cells with a stabilization buffer. In some embodiments, at least a subset of the plurality of cells are intact and inviable.

In further aspects, there are provided methods for preserving a population of cells. In some embodiments, the method comprises obtaining a vaginal fluid sample comprising a plurality of cells comprising trophoblast and trophoblast-derived cells, such as comprising at least one of a Hofbauer cell, a cytotrophoblast, a syncytiotrophoblast, an extravillous trophoblast, a villous core stroma, or a fetal vascular cell from a pregnant subject. In some embodiments, the method comprises placing the vaginal fluid sample in a stabilization buffer to create a mixture, wherein at least about 50% of the cells in the mixture are intact and inviable.

In additional aspects, there are provided methods for isolating a population of fetal trophoblast or other placental derived cells from a pregnant subject. In some embodiments, the method comprises: obtaining a mixture comprising (i) a vaginal fluid sample comprising a plurality of cells comprising trophoblast and trophoblast-derived cells, such as comprising at least one of a Hofbauer cell, a cytotrophoblast, a syncytiotrophoblast, an extravillous trophoblast, a villous core stroma, or a fetal vascular cell from the pregnant subject, and (ii) a stabilization buffer, wherein at least a subset of the plurality of cells are intact and inviable. In some embodiments, the method comprises, contacting the plurality of cells with at least one binding agent that specifically binds to a fetal cell surface protein and isolating cells bound to the binding agent, thereby isolating the population of fetal trophoblast or other placental derived cells. In some embodiments, the method comprises 2, 3, 4, 5, 6, 7, 8, 9, 10 rounds of maternal depletion. In some embodiments, the method comprises 2, 3, 4, 5, 6, 7, 8, 9, 10 rounds of fetal enrichment. In some embodiments, the method comprises 2, 3, 4, 5, 6, 7, 8, 9, or 10 rounds of maternal depletion followed by 2, 3, 4, 5, 6, 7, 8, 9, 10 rounds of fetal enrichment. In some embodiments, the method comprises 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 rounds of maternal depletion. In some embodiments, the method comprises 2 to 3, 2 to 4, 2 to 5, 2 to 6, 3 to 4, 3 to 5, 3 to 6, 4 to 5, 4 to 6, or 5 to 6 rounds of fetal enrichment. In some embodiments, the method comprises 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 rounds of maternal depletion followed by 2 to 3, 2 to 4, 2 to 5, 2 to 6, 3 to 4, 3 to 5, 3 to 6, 4 to 5, 4 to 6, or 5 to 6 rounds of fetal enrichment. In some embodiments, the method comprises 2 to 3, 2 to 4, 2 to 5, 2 to 6, 3 to 4, 3 to 5, 3 to 6, 4 to 5, 4 to 6, or 5 to 6 rounds of fetal enrichment followed by 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 rounds of maternal depletion. In some embodiments, the method comprises 4 rounds of maternal depletion followed by 2 rounds of fetal enrichment. In some embodiments, the method comprises 2 rounds of fetal enrichment followed by 4 rounds of maternal depletion. In some embodiments, the method comprises 4 rounds of maternal depletion followed by one round of FACS cell isolation using at least one fetal cell specific antibody.

In some embodiments, methods herein preserve cells such that at least 50% of the population of cells is intact. For example, in some embodiments, at least 55%, at least 60%, at least 65%, at least 70%, at least 75%, at least 80%, at least 85%, at least 90%, at least 95%, or more of the population of cells are intact. In some embodiments, at least 50% of the population of cells is intact after at least 1 day in the stabilization buffer. For example, in some embodiments, at least 50% of the population of cells is intact after at least 2 days, at least 3 days, at least 4 days, at least 5 days, at least 6 days, at least 7 days, at least 8 days, at least 9 days, at least 10 days, at least 11 days, at least 12 days, at least 13, days, at least 14 days, or more in the stabilization buffer. In some cases, at least 50% of the population of cells is intact after at least 1 day in the stabilization buffer at room temperature. In some cases, room temperature is about 10° C., about 15° C., about 20° C., about 25° C., about 30° C., or about 35° C. In some cases, room temperature is from about 10° C. to about 35° C., from about 10° C. to about 30° C., from about 10° C. to about 25° C., from about 10° C. to about 20° C., from about 10° C. to about 15° C., from about 15° C. to about 35° C., from about 15° C. to about 30° C., from about 15° C. to about 25° C., from about 15° C. to about 20° C., from about 20° C. to about 35° C., from about 20° C. to about 30° C., from about 20° C. to about 25° C., from about 25° C. to about 35° C., from about 25° C. to about 30° C., or from about 30° C. to about 35° C.

In some embodiments, methods herein utilize a stabilization buffer wherein the stabilization buffer maintains the size of the cells in the population as compared with the size of the cells prior to contact with the stabilization buffer. In some embodiments, the cells are maintained at a size at least 0.5× as compared with the size of the cells prior to contact with the stabilization buffer. For example, in some embodiments, cells are maintained at a size at least 0.6,×, at least 0.7×, at least 0.8×, at least 0.9×, or at least 1× as compared with the size of the cells prior to contact with the stabilization buffer. In some embodiments, cells are maintained at a size within a range of 0.5 to 1×, 0.5 to 0.9×, 0.5 to 0.8×, 0.5 to 0.7×, 0.5 to 0.6×, 0.6 to 1×, 0.6 to 0.9×, 0.6 to 0.8×, 0.6 to 0.7×, 0.7 to 1×, 0.7 to 0.9×, 0.7 to 0.8×, 0.8 to 1×, 0.8 to 0.9×, or 0.9 to 1× as compared with the size of the cells prior to contact with the stabilization buffer. In some embodiments, the stabilization buffer maintains the size of the cells in the population within a range of ±20% as compared with the size of the cells prior to contact with the stabilization buffer. In some embodiments, the stabilization buffer maintains the size of the cells in the population within a range of ±15% as compared with the size of the cells prior to contact with the stabilization buffer. In some embodiments, the stabilization buffer maintains the size of the cells in the population within a range of ±10% as compared with the size of the cells prior to contact with the stabilization buffer. In some embodiments, the stabilization buffer maintains the size of the cells in the population within a range of ±5% as compared with the size of the cells prior to contact with the stabilization buffer.

In some embodiments, methods herein result in a population of cells comprising trophoblast and trophoblast-derived cells, such as comprising a Hofbauer cell, a cytotrophoblast, a syncytiotrophoblast, an extravillous trophoblast, a villous core stroma, or a fetal vascular cell. In some embodiments, methods herein result in a population of cells comprising at least two of a Hofbauer cell, a cytotrophoblast, a syncytiotrophoblast, an extravillous trophoblast, a villous core stroma, or a fetal vascular cell. In some embodiments, methods herein result in a population of cells comprising at least three of a Hofbauer cell, a cytotrophoblast, a syncytiotrophoblast, an extravillous trophoblast, a villous core stroma, or a fetal vascular cell. In some embodiments, methods herein result in a population of cells comprising at least four of a Hofbauer cell, a cytotrophoblast, a syncytiotrophoblast, an extravillous trophoblast, a villous core stroma, or a fetal vascular cell. In some embodiments, methods herein result in a population of cells comprising at least five of a Hofbauer cell, a cytotrophoblast, a syncytiotrophoblast, an extravillous trophoblast, a villous core stroma, or a fetal vascular cell. In some embodiments, the population of cells comprises at least one of a cytotrophoblast or a syncytiotrophoblast. In some embodiments, the population of cells comprises at least one of a cytotrophoblast and a syncytiotrophoblast. In some embodiments, the method creates population enriched in cytotrophoblasts, syncytiotrophoblasts, extravillous trophoblasts, or any combination thereof.

In some embodiments of methods herein, vaginal fluid is collected using a cellulosic collection device. In some embodiments, the cellulosic collection device is placed in the vagina. In some embodiments, the cellulosic collection device is placed near the vagina, such as at or covering the vaginal opening. In some embodiments, the cellulosic collection device is a tampon, a pad, a plug, or a swab. In some embodiments, the cellulosic collection device is in or near the vagina for a suitable length of time to collect sufficient cells. For example, in some cases, the cellulosic collection device is in or near the vagina for at least 10, 20, 30, 40, 50, 60, 90, 120, 150, 180, 210, 240, 270, 300, 330, 360, 390, 420, 450, 480 minutes, or longer. In some embodiments, more than one cellulosic collection device is used to collect the vaginal fluid, for example 2, 3, or 4 cellulosic collection devices are used over the course of 1 day, 2 days, 3 days, 4 days, 5 days, 6 days, 1 week, 2 weeks, 3 weeks, 4 weeks, 8 weeks, 12 weeks, 16 weeks, 20 weeks, 24 weeks, 28 weeks, 32 weeks, 36 weeks, or 40 weeks in certain embodiments. In an embodiment, a fetal trophoblast composition is collected from a pregnant subject using 3 cellulosic collection devices over the course of a day. In an embodiment, fetal trophoblast compositions are collected over the course of a pregnancy, for example at 4 weeks, 8 weeks, 12 weeks, and 20 weeks gestation.

In some embodiments, methods herein further comprise contacting a population of cells to a binding agent that specifically binds to a cell surface marker. In some embodiments, methods herein further comprise isolating a subset of the plurality of cells that are bound to the binding agent. In some embodiments, the method comprises contacting the cells to a binding agent that binds to a maternal cell surface marker and the enriched population of depleted of maternal cells. In some embodiments, the method comprises contacting the plurality of cells with at least one binding agent that specifically binds to a fetal cell surface protein; and isolating cells bound to the binding agent, thereby isolating a population of fetal trophoblast or placental derived cells. In some embodiments, the method comprises contacting the plurality of cells with at least one binding agent that specifically binds to a maternal cell surface protein; and isolating cells bound to the binding agent, thereby isolating a population of maternal cells. In some embodiments, the cell surface marker is a fetal cell surface marker. In some embodiments, the cell surface marker is a maternal cell surface marker. In some embodiments, the cell surface marker comprises FSHR, CD45, LHCGR, CD270, Trop2, FOLR1, CD114 (CSF3R) or SLC40A1. In some embodiments, the cell surface marker comprises FSHR, CD45, LHCGR, CD270, CD156a, or ERVW1. In some embodiments, the cell surface marker comprises Trop2, GPC3, TREML2, ALPP (866), CD163, CD144, ALPP (GM022), FOLR1, CD114 (CSF3R), CD115, TSPAN1, SLC22A1, EFNA1, SLC62A2, SLC40A1, CD71, or CD166. In some embodiments, the cell surface marker is a fetal cell marker selected from Trop2, FOLR1, CD114 (CSF3R), or SLC40A1. In some embodiments, the cell surface marker is a maternal cell marker selected from FSHR, CD45, LHCGR, or CD270. In some cases, nuclei are isolated from the fetal trophoblast cells. In some embodiments, extravillous trophoblasts are enriched and the cell surface marker comprises at least one of TGF-beta2, human placental lactogen, c-erbB2, PAPPA2, or PRG2. In some embodiments, syncytiotrophoblasts are enriched and the cell surface marker comprises at least one of cytokeratin 7, beta-HCG, alpha-HCG, GCM1, syncytin, c-erbB2, leptin, INSL4, TGF-beta1, CSH1, or KISS1. In some embodiments, cytotrophoblsts are enriched and the cell surface marker comprises at least one of INSL4, PEG10, PAGE4, or p63.

In some embodiments of methods herein, the binding agent is an antibody or fragment thereof. In some embodiments, the binding agent comprises an IgG, IgD, IgM, IgA, or IgE. In some embodiments, the binding agent comprises an IgG1, and IgG2, IgG3 or IGg4. In some embodiments, the binding agent is a monoclonal antibody. In some embodiments, the binding agent is a polyclonal antibody. In some embodiments, the binding agent is an antigen binding fragment. In some embodiments, the binding agent is a Fab fragment, F(ab′)2 fragment, single chain Fv (scFv), diabody, triabody, or minibody. In some embodiments, the binding agent comprises a bead, such as a magnetic bead. In some embodiments, the binding agent comprises biotin or streptavidin. In some embodiments, the binding agent comprises a detectable label. In some embodiments, the detectable label comprises a fluorophore, such as a Fluorescein (FITC), an Allophycocyanin (APC), a R-Phycoerythrin (PE), a green fluorescent protein (GFP), a red fluorescent protein (RFP), a yellow fluorescent protein (YFP), or combination thereof.

In some embodiments, methods herein further comprise enriching for a population of cells including trophoblast and trophoblast-derived cells using methods such as density-gradient centrifugation, nuclei isolation and in some embodiments, such methods are combined with the use of a binding agent to deplete one or more cell types (such as maternal cells) and/or enrich for certain cell types such as fetal cells and populations of trophoblast and trophoblast-derived cells.

In some embodiments of methods herein, the population of cells comprises at least 500 cells. In some embodiments, the population of cells comprises at least 500 cells, at least 600 cells, at least 700 cells, at least 800 cells, at least 900 cells, at least 1000 cells, at least 1200 cells, at least 1500 cells, at least 1800 cells, at least 2000 cells, or more. In some embodiments, the population of cells comprises from about 500 cells to about 20000 cells, from about 500 cells to about 10000 cells, from about 500 cells to about 8000 cells, from about 500 cells to about 7500 cells, from about 500 cells to about 5000 cells, from about 500 cells to about 2500 cells, from about 500 cells to about 2000 cells, from about 500 cells to about 1800 cells, from about 500 cells to about 1500 cells, from about 500 cells to about 1200 cells, from about 500 cells to about 1000 cells, from about 500 cells to about 900 cells, from about 500 cells to about 800 cells, from about 500 cells to about 700 cells, or from about 500 cells to about 600 cells.

In some embodiments of methods herein, the population of enriched cells comprises between about 0.5% to 50% trophoblasts and trophoblast-derived cells. In some embodiments, the population of enriched cells comprises at least about 0.5%, at least about 1%, at least about 5%, at least about 10%, at least about 20%, at least about 30%, at least about 40%, or more trophoblasts and trophoblast-derived cells. In some embodiments, the population of enriched cells comprises at least about 0.5% to about 2% trophoblast and trophoblast-derived cells, at least about 1% to about 50% trophoblasts and trophoblast-derived cells, at least about 5% to about 10% trophoblasts and trophoblast-derived cells, at least about 10% to about 20% trophoblasts and trophoblast-derived cells, at least about 20% to about 30% trophoblasts and trophoblast-derived cells, at least about 30% to about 40% trophoblasts and trophoblast-derived cells, or at least about 40% to about 50% trophoblasts and trophoblast-derived cells. In some embodiments, the population of enriched cells comprises about 0.5% to 1%, 1% to 2%, 2% to 4%, 3% to 5%, 4% to 6%, 5% to 7%, 5% to 10%, 10% to 15%, 15% to 20%, 15% to 25%, 20% to 40%, 20% to 50%, 30% to 50%, 40% to 50% trophoblasts and trophoblast-derived cells. In some embodiments, the population of enriched cells comprises between about 50% to 99.5% maternal cells. For example, in some embodiments, the population of enriched cells comprises no more than about 99.5%, about 99%, about 90%, about 80% maternal cells, about 70% maternal cells, about 60% maternal cells, or about 50% maternal cells. In some embodiments, the population of enriched cells comprises no more than about 99-99.5%, 95-99%, 90-95%, 80-90%, 70-80%, 60-70%, or 50-60% maternal cells.

In some embodiments, the population of enriched cells comprises at least 50% trophoblasts and trophoblast-derived cells. In some embodiments, the population of enriched cells comprises at least about 50%, at least about 60%, at least about 70%, at least about 80%, or more trophoblasts and trophoblast-derived cells. In some embodiments, the population of enriched cells comprises at least about 40% to about 50% trophoblast, at least about 50% to about 60% trophoblasts and trophoblast-derived cells, at least about 60% to about 70% trophoblasts and trophoblast-derived cells, at least about 70% to about 80% trophoblasts and trophoblast-derived cells, at least about 50% to about 70% trophoblasts and trophoblast-derived cells, at least about 50% to about 80% trophoblasts and trophoblast-derived cells, or at least about 60% to about 80% trophoblasts and trophoblast-derived cells. In some embodiments, the population of enriched cells comprises about 50% to 60%, 50% to 70%, 50% to 80%, 50% to 90%, 50% to 95%, 50% to 99%, 60% to 70%, 60% to 80%, 60% to 90%, 60% to 95%, 60% to 99%, 70% to 80%, 70% to 90%, 70% to 95%, 70% to 99%, 80% to 90%, 80% to 95%, 80% to 99%, 90% to 95%, 90% to 99%, or 95% to 99% trophoblasts and trophoblast-derived cells. In some embodiments, the population of enriched cells comprises no more than about 50% maternal cells. For example, in some embodiments, the population of enriched cells comprises no more than about 50%, about 40%, about 30%, about 20% maternal cells, about 10% maternal cells, about 5% maternal cells, or about 1% maternal cells. In some embodiments, the population of enriched cells comprises no more than about 1% to 5%, 1% to 10%, 1% to 20%, 1% to 30%, 1% to 40%, 1% to 50%, 5% to 10%, 5% to 20%, 5% to 30%, 5% to 40%, 5% to 50%, 10% to 20%, 10% to 30%, 10% to 40%, 10% to 50%, 20% to 30%, 20% to 40%, 20% to 50%, 30% to 40%, 30% to 50%, or 30% to 50% maternal cells. In some embodiments, a trophoblast composition has a fetal trophoblast to background ratio of 1/400 prior to enrichment.

In some embodiments of methods herein, the method further comprises quantifying a fetal fraction of the plurality of cells. In some embodiments, quantifying a fetal fraction comprises cellular staining, intracellular staining, RNA analysis, short tandem repeat (STR) analysis, single nucleotide polymorphism (SNP) analysis, Y-chromosome analysis, RNaseP analysis, or combinations thereof. In some embodiments, the fetal fraction is quantified using miRNA expression, for example one or more of miR-1246, miR-1323, miR-512-3p, hsa-miR-516b-5p, miR-517-5p, hsa-miR-525, miR-526b, ath-miR159a, RNU44, RNU48, or U6.

In some embodiments of methods herein, cells are contacted to a stabilization buffer. In some embodiments, the stabilization buffer comprises at least one of: a preservation agent, a dissociation agent, or a combination thereof. In some embodiments, the stabilization buffer comprises a zwitterionic compound, an osmoprotectant, an apoptosis inhibitor, a non-reducing sugar or polyol, a disaccharide derivative, a chelating agent, a pH buffer, a phosphatase inhibitor, a protease inhibitor, or a combination thereof. In some embodiments, the dissociation agent comprises a mucolytic, an expectorant, a surfactant, a nuclease, a protease, or a combination thereof. In some embodiments, the stabilization buffer comprises a zwitterionic compound, an osmoprotectant, an apoptosis inhibitor, a non-reducing sugar or polyol, a chelating agent, a pH buffer, a phosphatase inhibitor, a protease inhibitor, a mucolytic, an expectorant, a surfactant, a nuclease, a protease, or any combination thereof. In some embodiments, the stabilization buffer comprises phosphate buffered saline, ethylenediaminetetraacetic acid, fetal bovine serum, Hank's balanced salt solution, collagenase, accutase, a nuclease, or combinations thereof. Additional components of stabilization buffers are provided elsewhere herein.

In some embodiments of methods herein, the method further comprises removing mucus from the plurality of cells. In some embodiments, mucus is removed using a nuclease. In some embodiments, mucus is removed using a protease. In some embodiments, mucus is removed using a nuclease and a protease. In some embodiments, mucus is removed using a surfactant. In some embodiments, mucus is removed using a mucolytic agent. In some embodiments, mucus is removed using a nuclease, a protease, a surfactant, a mucolytic agent, or a combination thereof.

In some embodiments of methods herein, the method further comprises analyzing at least one of a nucleic acid, a protein, a carbohydrate, or a lipid from the plurality of cells. In some embodiments, analyzing comprises one or more of PCR, sequencing, karyotyping, in situ hybridization, immunofluorescence, FACS, or mass spectroscopy. For example, in some cases, nucleic acids are isolated from the cells and PCR, such as quantitative PCR is used to determine a copy number or an expression level for a locus of interest. In another non-limiting example, PCR is used to identify a specific locus. In some cases, sequencing is used to determine a sequence for the isolated nucleic acids or a subset thereof to identify a copy number variant, a single nucleotide variant, an insertion, a deletion, an epigenetic modification, or a combination thereof. In some cases, sequencing is used to identify an epigenetic modification such as methylation, phosphorylation, ubiquitination, sumoylation, acetylation, ribosylation, citrullination, or a combination thereof. In some cases, nucleic acids in the cells are stained with a dye and fluorescent in situ hybridization (FISH) is used to analyze the chromosomes or alternatively a karyotype is created from the cells. In some cases, a panel of cell surface or intracellular markers is studied using immunofluorescence. In some cases, mass spectroscopy is used to create a carbohydrate or lipid profile for the cells.

In some embodiments of methods herein, the method further comprises recommending a treatment to the pregnant subject. In some embodiments, the method further comprises recommending additional testing to the pregnant subject. In some embodiments, the treatment comprises administration of a recombinant protein when a genetic deficiency is found. For example, in some cases the fetal trophoblast cells are found to carry X-Linked Hypohidrotic Ectodermal Dysplasia and recombinant ectodysplasin A is administered in utero to treat the fetus in utero.

### Methods for Fetal Cell Analysis

In additional aspects, there are provided, methods for fetal cell analysis. In some embodiments, methods comprise isolating a population of fetal trophoblast or other placental derived cells (i.e., trophoblast and trophoblast-derived cells) comprising at least one of a Hofbauer cell, a cytotrophoblast, a syncytiotrophoblast, an extravillous trophoblast, a villous core stroma, or a fetal vascular cell from a vaginal fluid sample from a pregnant subject wherein at least a subset of the population of cells are intact and inviable. In some embodiments, methods comprise obtaining fetal nucleic acids from the population of fetal cells. In some embodiments, methods comprise analyzing at least one feature of the fetal nucleic acids.

In some embodiments of methods herein, the method further comprises quantifying a fetal fraction of the plurality of cells. In some embodiments, quantifying a fetal fraction comprises cellular staining, intracellular staining, RNA analysis, short tandem repeat (STR) analysis, single nucleotide polymorphism (SNP) analysis, Y-chromosome analysis, RNaseP analysis, or combinations thereof. In some embodiments, the fetal fraction is quantified using miRNA expression, for example one or more of miR-1246, miR-1323, miR-512-3p, hsa-miR-516b-5p, miR-517-5p, hsa-miR-525, miR-526b, ath-miR159a, RNU44, RNU48, or U6.

In some embodiments, methods herein result in an enriched population of cells comprising trophoblast and trophoblast-derived cells) such as a Hofbauer cell, a cytotrophoblast, a syncytiotrophoblast, an extravillous trophoblast, a villous core stroma, or a fetal vascular cell. In some embodiments, methods herein result in an enriched population of cells comprising at least two of a Hofbauer cell, a cytotrophoblast, a syncytiotrophoblast, an extravillous trophoblast, a villous core stroma, or a fetal vascular cell. In some embodiments, methods herein result in an enriched population of cells comprising at least three of a Hofbauer cell, a cytotrophoblast, a syncytiotrophoblast, an extravillous trophoblast, a villous core stroma, or a fetal vascular cell. In some embodiments, methods herein result in an enriched population of cells comprising at least four of a Hofbauer cell, a cytotrophoblast, a syncytiotrophoblast, an extravillous trophoblast, a villous core stroma, or a fetal vascular cell. In some embodiments, methods herein result in an enriched population of cells comprising at least five of a Hofbauer cell, a cytotrophoblast, a syncytiotrophoblast, an extravillous trophoblast, a villous core stroma, or a fetal vascular cell. In some embodiments, the enriched population of cells comprises at least one of a cytotrophoblast or a syncytiotrophoblast. In some embodiments, the enriched population of cells comprises at least one of a cytotrophoblast and a syncytiotrophoblast. In some embodiments, the method creates population enriched in cytotrophoblasts, syncytiotrophoblasts, extravillous trophoblasts, or any combination thereof.

In some embodiments, methods herein further comprise contacting a population of cells to a binding agent that specifically binds to a cell surface marker. In some embodiments, methods herein further comprise isolating a subset of the plurality of cells that are bound to the binding agent. In some embodiments, the method comprises contacting the cells to a binding agent that binds to a maternal cell surface marker and the enriched population of depleted of maternal cells. In some embodiments, the method comprises contacting the plurality of cells with at least one binding agent that specifically binds to a fetal cell surface protein; and isolating cells bound to the binding agent, thereby isolating a population of fetal trophoblast or other placental derived cells. In some embodiments, the method comprises contacting the plurality of cells with at least one binding agent that specifically binds to a maternal cell surface protein; and isolating cells bound to the binding agent, thereby isolating a population of maternal cells. In some embodiments, the cell surface marker is a fetal cell surface marker. In some embodiments, the cell surface marker is a maternal cell surface marker. In some embodiments, the cell surface marker comprises FSHR, CD45, LHCGR, CD270, Trop2, FOLR1, CD114 (CSF3R) or SLC40A1. In some embodiments, the cell surface marker comprises FSHR, CD45, LHCGR, CD270, CD156a, or ERVW1. In some embodiments, the cell surface marker comprises Trop2, GPC3, TREML2, ALPP (866), CD163, CD144, ALPP (GM022), FOLR1, CD114 (CSF3R), CD115, TSPAN1, SLC22A1, EFNA1, SLC62A2, SLC40A1, CD71, or CD166. In some embodiments, the cell surface marker is a fetal cell marker selected from Trop2, FOLR1, CD114 (CSF3R), or SLC40A1. In some embodiments, the cell surface marker is a maternal cell marker selected from FSHR, CD45, LHCGR, or CD270. In some embodiments, extravillous trophoblasts are enriched and the cell surface marker comprises at least one of TGF-beta2, human placental lactogen, c-erbB2, PAPPA2, or PRG2. In some embodiments, syncytiotrophoblasts are enriched and the cell surface marker comprises at least one of cytokeratin 7, beta-HCG, alpha-HCG, GCM1, syncytin, c-erbB2, leptin, INSL4, TGF-beta1, CSH1, or KISS1. In some embodiments, cytotrophoblsts are enriched and the cell surface marker comprises at least one of INSL4, PEG10, PAGE4, or p63.

In some embodiments of methods herein, the binding agent is an antibody or fragment thereof. In some embodiments, the binding agent comprises an IgG, IgD, IgM, IgA, or IgE. In some embodiments, the binding agent comprises an IgG1, and IgG2, IgG3 or IGg4. In some embodiments, the binding agent is a monoclonal antibody. In some embodiments, the binding agent is a polyclonal antibody. In some embodiments, the binding agent is an antigen binding fragment. In some embodiments, the binding agent is a Fab fragment, F(ab′)2 fragment, single chain Fv (scFv), diabody, triabody, or minibody. In some embodiments, the binding agent comprises a bead, such as a magnetic bead. In some embodiments, the binding agent comprises biotin or streptavidin. In some embodiments, the binding agent comprises a detectable label. In some embodiments, the detectable label comprises a fluorophore, such as a Fluorescein (FITC), an Allophycocyanin (APC), a R-Phycoerythrin (PE), a green fluorescent protein (GFP), a red fluorescent protein (RFP), a yellow fluorescent protein (YFP), or combination thereof.

In some embodiments, methods herein further comprise enriching for a population of cells including trophoblast and trophoblast-derived cells using methods such as density-gradient centrifugation, nuclei isolation and in some embodiments, such methods are combined with the use of a binding agent to deplete one or more cell types (such as maternal cells) and/or enrich for certain cell types such as fetal cells and populations of trophoblast and trophoblast-derived cells.

In some embodiments of methods herein, the method further comprises analyzing at least one of a nucleic acid, a protein, a carbohydrate, or a lipid from the plurality of cells. In some embodiments, analyzing comprises one or more of PCR, sequencing, karyotyping, in situ hybridization, immunofluorescence, FACS, or mass spectroscopy. For example, in some cases, nucleic acids are isolated from the cells and PCR, such as quantitative PCR is used to determine a copy number for a locus of interest. In another non-limiting example, PCR is used to identify a specific locus. In some cases, sequencing is used to determine a sequence for the isolated nucleic acids or a subset thereof to identify a copy number variant, a single nucleotide variant, an insertion, a deletion, an epigenetic modification, or a combination thereof. In some cases, sequencing is used to identify an epigenetic modification such as methylation, phosphorylation, ubiquitination, sumoylation, acetylation, ribosylation, citrullination, or a combination thereof. In some cases, nucleic acids in the cells are stained with a dye and fluorescent in situ hybridization (FISH) is used to analyze the chromosomes or alternatively a karyotype is created from the cells. In some cases, a panel of cell surface or intracellular markers is studied using immunofluorescence. In some cases, mass spectroscopy is used to create a carbohydrate or lipid profile for the cells.

In some embodiments, methods herein further comprise contacting cells to a stabilization buffer that preserves cells such that at least 50% of the population of cells is intact. For example, in some embodiments, at least 55%, at least 60%, at least 65%, at least 70%, at least 75%, at least 80%, at least 85%, at least 90%, at least 95%, or more of the population of cells are intact. In some embodiments, at least 50% of the population of cells is intact after at least 1 day in the stabilization buffer. For example, in some embodiments, at least 50% of the population of cells is intact after at least 2 days, at least 3 days, at least 4 days, at least 5 days, at least 6 days, at least 7 days, at least 8 days, at least 9 days, at least 10 days, at least 11 days, at least 12 days, at least 13, days, at least 14 days, or more in the stabilization buffer. In some cases, at least 50% of the population of cells is intact after at least 1 day in the stabilization buffer at room temperature. In some cases, room temperature is about 10° C., about 15° C., about 20° C., about 25° C., about 30° C., or about 35° C. In some cases, room temperature is from about 10° C. to about 35° C., from about 10° C. to about 30° C., from about 10° C. to about 25° C., from about 10° C. to about 20° C., from about 10° C. to about 15° C., from about 15° C. to about 35° C., from about 15° C. to about 30° C., from about 15° C. to about 25° C., from about 15° C. to about 20° C., from about 20° C. to about 35° C., from about 20° C. to about 30° C., from about 20° C. to about 25° C., from about 25° C. to about 35° C., from about 25° C. to about 30° C., or from about 30° C. to about 35° C.

In some embodiments, methods herein utilize a stabilization buffer wherein the stabilization buffer maintains the size of the cells in the population as compared with the size of the cells prior to contact with the stabilization buffer. In some embodiments, the cells are maintained at a size at least 0.5× as compared with the size of the cells prior to contact with the stabilization buffer. For example, in some embodiments, cells are maintained at a size at least 0.6,×, at least 0.7×, at least 0.8×, at least 0.9×, or at least 1× as compared with the size of the cells prior to contact with the stabilization buffer. In some embodiments, cells are maintained at a size within a range of 0.5 to 1×, 0.5 to 0.9×, 0.5 to 0.8×, 0.5 to 0.7×, 0.5 to 0.6×, 0.6 to 1×, 0.6 to 0.9×, 0.6 to 0.8×, 0.6 to 0.7×, 0.7 to 1×, 0.7 to 0.9×, 0.7 to 0.8×, 0.8 to 1×, 0.8 to 0.9×, or 0.9 to 1× as compared with the size of the cells prior to contact with the stabilization buffer. In some embodiments, the stabilization buffer maintains the size of the cells in the population within a range of ±20% as compared with the size of the cells prior to contact with the stabilization buffer. In some embodiments, the stabilization buffer maintains the size of the cells in the population within a range of ±15% as compared with the size of the cells prior to contact with the stabilization buffer. In some embodiments, the stabilization buffer maintains the size of the cells in the population within a range of ±10% as compared with the size of the cells prior to contact with the stabilization buffer. In some embodiments, the stabilization buffer maintains the size of the cells in the population within a range of ±5% as compared with the size of the cells prior to contact with the stabilization buffer.

In some embodiments of methods herein, cells are contacted to a stabilization buffer. In some embodiments, the stabilization buffer comprises at least one of: a preservation agent, a dissociation agent, or a combination thereof. In some embodiments, the stabilization buffer comprises a zwitterionic compound, an osmoprotectant, an apoptosis inhibitor, a non-reducing sugar or polyol, a disaccharide derivative, a chelating agent, a pH buffer, a phosphatase inhibitor, a protease inhibitor, or a combination thereof. In some embodiments, the dissociation agent comprises a mucolytic, an expectorant, a surfactant, a nuclease, a protease, or a combination thereof. In some embodiments, the stabilization buffer comprises a zwitterionic compound, an osmoprotectant, an apoptosis inhibitor, a non-reducing sugar or polyol, a chelating agent, a pH buffer, a phosphatase inhibitor, a protease inhibitor, a mucolytic, an expectorant, a surfactant, a nuclease, a protease, or any combination thereof. In some embodiments, the stabilization buffer comprises phosphate buffered saline, ethylenediaminetetraacetic acid, fetal bovine serum, Hank's balanced salt solution, collagenase, accutase, a nuclease, or combinations thereof. Additional components of stabilization buffers are provided elsewhere herein.

In some embodiments of methods herein, the population of cells comprises at least 500 cells. In some embodiments, the population of cells comprises at least 500 cells, at least 600 cells, at least 700 cells, at least 800 cells, at least 900 cells, at least 1000 cells, at least 1200 cells, at least 1500 cells, at least 1800 cells, at least 2000 cells, or more. In some embodiments, the population of cells comprises from about 500 cells to about 20000 cells, from about 500 cells to about 10000 cells, from about 500 cells to about 8000 cells, from about 500 cells to about 7500 cells, from about 500 cells to about 5000 cells, from about 500 cells to about 2500 cells, from about 500 cells to about 2000 cells, from about 500 cells to about 1800 cells, from about 500 cells to about 1500 cells, from about 500 cells to about 1200 cells, from about 500 cells to about 1000 cells, from about 500 cells to about 900 cells, from about 500 cells to about 800 cells, from about 500 cells to about 700 cells, or from about 500 cells to about 600 cells.

In some embodiments of methods herein, the population of enriched cells comprises between about 0.5% to 50% trophoblasts and trophoblast-derived cells. In some embodiments, the population of enriched cells comprises at least about 0.5%, at least about 1%, at least about 5%, at least about 10%, at least about 20%, at least about 30%, at least about 40%, or more trophoblasts and trophoblast-derived cells. In some embodiments, the population of enriched cells comprises at least about 0.5% to about 2% trophoblast and trophoblast-derived cells, at least about 1% to about 50% trophoblasts and trophoblast-derived cells, at least about 5% to about 10% trophoblasts and trophoblast-derived cells, at least about 10% to about 20% trophoblasts and trophoblast-derived cells, at least about 20% to about 30% trophoblasts and trophoblast-derived cells, at least about 30% to about 40% trophoblasts and trophoblast-derived cells, or at least about 40% to about 50% trophoblasts and trophoblast-derived cells. In some embodiments, the population of enriched cells comprises about 0.5% to 1%, 1% to 2%, 2% to 4%, 3% to 5%, 4% to 6%, 5% to 7%, 5% to 10%, 10% to 15%, 15% to 20%, 15% to 25%, 20% to 40%, 20% to 50%, 30% to 50%, 40% to 50% trophoblasts and trophoblast-derived cells. In some embodiments, the population of enriched cells comprises between about 50% to 99.5% maternal cells. For example, in some embodiments, the population of enriched cells comprises no more than about 99.5%, about 99%, about 90%, about 80% maternal cells, about 70% maternal cells, about 60% maternal cells, or about 50% maternal cells. In some embodiments, the population of enriched cells comprises no more than about 99-99.5%, 95-99%, 90-95%, 80-90%, 70-80%, 60-70%, or 50-60% maternal cells.

In some embodiments, the population of enriched cells comprises at least 50% trophoblasts and trophoblast-derived cells. In some embodiments, the population of enriched cells comprises at least about 50%, at least about 60%, at least about 70%, at least about 80%, or more trophoblasts and trophoblast-derived cells. In some embodiments, the population of enriched cells comprises at least about 40% to about 50% trophoblast and trophoblast-derived cells, at least about 50% to about 60% trophoblasts and trophoblast-derived cells, at least about 60% to about 70% trophoblasts and trophoblast-derived cells, at least about 70% to about 80% trophoblasts and trophoblast-derived cells, at least about 50% to about 70% trophoblasts and trophoblast-derived cells, at least about 50% to about 80% trophoblasts and trophoblast-derived cells, or at least about 60% to about 80% trophoblasts and trophoblast-derived cells. In some embodiments, the population of cells comprises about 50% to 60%, 50% to 70%, 50% to 80%, 50% to 90%, 50% to 95%, 50% to 99%, 60% to 70%, 60% to 80%, 60% to 90%, 60% to 95%, 60% to 99%, 70% to 80%, 70% to 90%, 70% to 95%, 70% to 99%, 80% to 90%, 80% to 95%, 80% to 99%, 90% to 95%, 90% to 99%, or 95% to 99% trophoblasts and trophoblast-derived cells. In some embodiments, the population of enriched cells comprises no more than about 50% maternal cells. For example, in some embodiments, the population of enriched cells comprises no more than about 50%, about 40%, about 30%, about 20% maternal cells, about 10% maternal cells, about 50% maternal cells, or about 1% maternal cells. In some embodiments, the population of cells comprises no more than about 1% to 5%, 1% to 10%, 1% to 20%, 1% to 30%, 1% to 40%, 1% to 50%, 5% to 10%, 5% to 20%, 5% to 30%, 5% to 40%, 5% to 50%, 10% to 20%, 10% to 30%, 10% to 40%, 10% to 50%, 20% to 30%, 20% to 40%, 20% to 50%, 30% to 40%, 30% to 50%, or 30% to 50% maternal cells. In some embodiments, a trophoblast composition has a fetal trophoblast to background ratio of 1/400 prior to enrichment.

In some embodiments of methods herein, vaginal fluid is collected using a cellulosic collection device. In some embodiments, the cellulosic collection device is placed in the vagina. In some embodiments, the cellulosic collection device is placed near the vagina, such as at or covering the vaginal opening. In some embodiments, the cellulosic collection device is a tampon, a pad, a plug, or a swab. In some embodiments, the cellulosic collection device is in or near the vagina for a suitable length of time to collect sufficient cells. For example, in some cases, the cellulosic collection device is in or near the vagina for at least 10, 20, 30, 40, 50, 60, 90, 120, 150, 180, 210, 240, 270, 300, 330, 360, 390, 420, 450, 480 minutes, or longer. In some embodiments, more than one cellulosic collection device is used to collect the vaginal fluid, for example 2, 3, or 4 cellulosic collection devices are used over the course of 1 day, 2 days, 3 days, 4 days, 5 days, 6 days, 1 week, 2 weeks, 3 weeks, 4 weeks, 8 weeks, 12 weeks, 16 weeks, 20 weeks, 24 weeks, 28 weeks, 32 weeks, 36 weeks, or 40 weeks in certain embodiments. In an embodiment, a pregnant subject uses 3 cellulosic collection devices over the course of a day to collect fetal trophoblast cells for analysis. In an embodiment, a fetal trophoblast cells are collected from a pregnant subject over the course of a pregnancy, for example at 4 weeks, 8 weeks, 12 weeks, and 20 weeks gestation.

In some embodiments of methods herein, the method further comprises removing mucus from the plurality of cells. In some embodiments, mucus is removed using a nuclease. In some embodiments, mucus is removed using a protease. In some embodiments, mucus is removed using a nuclease and a protease. In some embodiments, mucus is removed using a surfactant. In some embodiments, mucus is removed using a mucolytic agent. In some embodiments, mucus is removed using a nuclease, a protease, a surfactant, a mucolytic agent, or a combination thereof.

In some embodiments of methods herein, the method further comprises recommending a treatment to the pregnant subject. In some embodiments, the method further comprises recommending additional testing to the pregnant subject. In some embodiments, the treatment comprises administration of a recombinant protein when a genetic deficiency is found. For example, in some cases the fetal trophoblast cells are found to carry X-Linked Hypohidrotic Ectodermal Dysplasia and recombinant ectodysplasin A is administered in utero to treat the fetus in utero.

### EMBODIMENTS

1. A composition comprising: (a) a population of cells comprising at least one of a Hofbauer cell, a cytotrophoblast, a syncytiotrophoblast, an extravillous trophoblast, a villous core stroma, or a fetal vascular cell; (b) a stabilization buffer, wherein the stabilization buffer comprises a preservative; and (c) a cellulosic collection device, wherein at least a subset of the population of cells are intact and inviable.

2. The composition of embodiment 1, wherein the cellulosic collection device comprises a tampon, a pad, a plug, or a swab.

3. The composition of embodiment 1, wherein at least a portion of the population of cells are associated with the cellulosic collection device.

4. The composition of embodiment 1, wherein at least 50%, at least 60%, or at least 70% of the population of cells are intact after 1, 2, 3, 4, 5, 6, 7, 8, 9, or 10 days in the stabilization buffer at room temperature.

5. The composition of embodiment 1, wherein the stabilization buffer maintains the size of the cells in the population within a range of 0.5 to 1× as compared with the size of the cells prior to contact with the stabilization buffer.

6. The composition of embodiment 5, wherein the stabilization buffer maintains the size of the cells in the population within a range of ±20%, ±10%, or ±5% as compared with the size of the cells prior to contact with the stabilization buffer.

7. The composition of embodiment 1, wherein the population of cells comprises at least two of a Hofbauer cell, a cytotrophoblast, a syncytiotrophoblast, an extravillous trophoblast, a villous core stroma, or a fetal vascular cell.

8. The composition of embodiment 1, wherein the population of cells comprises at least one of a cytotrophoblast or a syncytiotrophoblast.

9. The composition of embodiment 1, further comprising a binding agent that specifically binds to a cell surface marker.

10. The composition of embodiment 9, wherein the cell surface marker is a fetal cell surface marker.

11. The composition of embodiment 9, wherein the cell surface marker is a maternal cell surface marker.

12. The composition of embodiment 9, wherein the cell surface marker comprises FSHR, CD45, LHCGR, CD270, Trop2, FOLR1, CD114 (CSF3R) or SLC40A1.

13. The composition of embodiment 9, wherein the binding agent is an antibody or fragment thereof.

14. The composition of embodiment 9, wherein the binding agent comprises a magnetic bead.

15. The composition of embodiment 9, wherein the binding agent comprises a detectable label.

16. The composition of embodiment 1, wherein the population of cells comprises at least 100 cells or at least 500 cells.

17. The composition of embodiment 1, wherein the population of cells comprises at least 50% trophoblasts.

18. The composition of embodiment 1, wherein the stabilization buffer comprises phosphate buffered saline, ethylenediaminetetraacetic acid, fetal bovine serum, Hank's balanced salt solution, collagenase, accutase, or a nuclease.

19. The composition of embodiment 1, wherein the stabilization buffer comprises a salt solution, a chelating agent, serum, an enzymic tissue, a nucleic acid active agent or any combinations thereof.

20. A composition comprising: (a) a population of cells comprising at least one of a Hofbauer cell, a cytotrophoblast, a syncytiotrophoblast, an extravillous trophoblast, a villous core stroma, or a fetal vascular cell; and (b) a stabilization buffer, wherein the stabilization buffer comprises a preservative and wherein at least a subset of the population of cells are intact and inviable.

21. The composition of embodiment 20, wherein at least 50%, at least 60%, or at least 70% of the population of cells are intact after 1, 2, 3, 4, 5, 6, 7, 8, 9, or 10 days in the stabilization buffer at room temperature.

22. The composition of embodiment 20, wherein the stabilization buffer maintains the size of the cells in the population within a range of 0.5 to 1× as compared with the size of the cells prior to contact with the stabilization buffer.

23. The composition of embodiment 22, wherein the stabilization buffer maintains the size of the cells in the population within a range of ±20%, ±10%, or ±5% as compared with the size of the cells prior to contact with the stabilization buffer.

24. The composition of embodiment 20, wherein the population of cells comprises at least two of a Hofbauer cell, a cytotrophoblast, a syncytiotrophoblast, an extravillous trophoblast, a villous core stroma, or a fetal vascular cell.

25. The composition of embodiment 20, wherein the population of cells comprises at least one of a cytotrophoblast or a syncytiotrophoblast.

26. The composition of embodiment 20, further comprising a binding agent that specifically binds to a cell surface marker.

27. The composition of embodiment 26, wherein the cell surface marker is a fetal cell surface marker.

28. The composition of embodiment 26, wherein the cell surface marker is a maternal cell surface marker.

29. The composition of embodiment 26, wherein the cell surface marker comprises FSHR, CD45, LHCGR, CD270, Trop2, FOLR1, CD114 (CSF3R) or SLC40A1.

30. The composition of embodiment 26, wherein the binding agent is an antibody or fragment thereof.

31. The composition of embodiment 26, wherein the binding agent comprises a magnetic bead.

32. The composition of embodiment 26, wherein the binding agent comprises a detectable label.

33. The composition of embodiment 20, wherein the population of cells comprises at least 500 cells.

34. The composition of embodiment 20, wherein the population of cells comprises at least 50% trophoblasts.

35. The composition of embodiment 20, wherein the stabilization buffer comprises phosphate buffered saline, ethylenediaminetetraacetic acid, fetal bovine serum, Hank's balanced salt solution, collagenase, accutase, or a nuclease.

36. The composition of embodiment 20, further comprising a cellulosic collection device.

37. The composition of embodiment 36, wherein the cellulosic collection device is a tampon, a pad, a plug, or a swab.

38. A composition comprising: (a) an enriched population of intact and nonviable trophoblast cells, wherein the population of cells comprises between 0.5% to 50% trophoblast and trophoblast-derived cells and at least 50%, at least 60%, or at least 70% of the enriched population are intact cells.

39. The composition of embodiment 38 wherein the population of cells comprises at least 0.5%, 1%, 2%, 5%, 10%, 20%, 25%, 30%, 40% or 50% trophoblast and trophoblast-derived cells.

40. The composition of embodiment 38 wherein the population of cells is enriched for trophoblast and trophoblast-derived cells between about 2-fold and 2000-fold.

41. The composition of embodiment 38 wherein the population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold.

42. The composition of embodiment 38, wherein the enriched population of cells are intact after 1, 2, 3, 4, 5, 6, 7, 8, 9, or 10 days in the stabilization buffer at room temperature.

43. The composition of embodiment 38, wherein the stabilization buffer maintains the size of the cells in the enriched population within a range of 0.5 to 1× as compared with the size of the cells prior to contact with the stabilization buffer.

44. The composition of embodiment 43, wherein the stabilization buffer maintains the size of the cells in the enriched population within a range of ±20%, ±10%, or ±5% as compared with the size of the cells prior to contact with the stabilization buffer.

45. The composition of embodiment 38, wherein the enriched population of cells comprises at least two cell types selected from the group consisting of a cytotrophoblast, a syncytiotrophoblast, and extravillous trophoblast.

46. The composition of embodiment 38, wherein the enriched population of cells comprises at least cytotrophoblasts or syncytiotrophoblasts.

47. The composition of embodiment 38, further comprising a binding agent that specifically binds to a cell surface marker.

48. The composition of embodiment 47, wherein the cell surface marker is a fetal cell surface marker.

49. The composition of embodiment 47, wherein the cell surface marker comprises FSHR, CD45, LHCGR, CD270, Trop2, FOLR1, CD114 (CSF3R) or SLC40A1.

50. The composition of embodiment 47, wherein the binding agent is an antibody or fragment thereof.

51. The composition of embodiment 47, wherein the binding agent comprises a magnetic bead.

52. The composition of embodiment 47, wherein the binding agent comprises a detectable label.

53. The composition of embodiment 38, wherein the enriched population of cells comprises at least 500 cells.

54. The composition of embodiment 38, wherein the enriched population of cells comprises at least 50% trophoblasts.

55. The composition of embodiment 38, wherein the stabilization buffer comprises phosphate buffered saline, ethylenediaminetetraacetic acid, fetal bovine serum, Hank's balanced salt solution, collagenase, accutase, or a nuclease.

56. The composition of embodiment 38, further comprising a cellulosic collection device.

57. The composition of embodiment 38, wherein the cellulosic collection device is a tampon, a pad, a plug, or a swab.

58. A composition comprising (a) a population of cells comprising at least one of a Hofbauer cell, a cytotrophoblast, a syncytiotrophoblast, an extravillous trophoblast, a villous core stroma, or a fetal vascular cell; and (b) one or more binding agents, wherein the one or more binding agents is capable of specifically binding to a cell surface protein.

59. The composition of embodiment 58, wherein the cell surface marker is a fetal cell surface marker.

60. The composition of embodiment 58, wherein the cell surface marker is a maternal cell surface marker.

61. The composition of embodiment 58, wherein the cell surface marker comprises FSHR, CD45, LHCGR, CD270, Trop2, FOLR1, CD114 (CSF3R) or SLC40A1.

62. The composition of embodiment 58, wherein the binding agent is an antibody or fragment thereof.

63. The composition of embodiment 58, wherein the binding agent comprises a magnetic bead.

64. The composition of embodiment 58, wherein the binding agent comprises a detectable label.

65. The composition of embodiment 58, wherein the population of cells comprises at least two of a Hofbauer cell, a cytotrophoblast, a syncytiotrophoblast, an extravillous trophoblast, a villous core stroma, or a fetal vascular cell.

66. The composition of embodiment 58, wherein the population of cells comprises at least one of a cytotrophoblast or a syncytiotrophoblast.

67. The composition of embodiment 58, further comprising a stabilization buffer.

68. The composition of embodiment 67, wherein at least 50%, at least 60%, or at least 70% of the population of cells are intact after 1, 2, 3, 4, 5, 6, 7, 8, 9, or 10 days in the stabilization buffer at room temperature.

69. The composition of embodiment 67, wherein the stabilization buffer maintains the size of the cells in the population within a range of 0.5 to 1× as compared with the size of the cells prior to contact with the stabilization buffer.

70. The composition of embodiment 69, wherein the stabilization buffer maintains the size of the cells in the population within a range of ±20%, ±10%, or ±5% as compared with the size of the cells prior to contact with the stabilization buffer.

71. The composition of embodiment 67, wherein the stabilization buffer comprises phosphate buffered saline, ethylenediaminetetraacetic acid, fetal bovine serum, Hank's balanced salt solution, collagenase, accutase, or a nuclease.

72. The composition of embodiment 58, further comprising a cellulosic collection device.

73. The composition of embodiment 72, wherein the cellulosic collection device is a tampon, a pad, a plug, or a swab.

74. The composition of embodiment 58, wherein the population of cells comprises at least 500 cells.

75. The composition of embodiment 58, wherein the population of cells comprises at least 50% trophoblasts.

76. A method of obtaining a stabilized population of cells, the method comprising: (a) placing a cellulosic collection device in or near a vagina of a pregnant subject to collect vaginal fluid comprising a plurality of cells comprising at least one of a Hofbauer cell, a cytotrophoblast, a syncytiotrophoblast, an extravillous trophoblast, a villous core stroma, or a fetal vascular cell; and (b) contacting the cellulosic collection device comprising the plurality of cells with a stabilization buffer, wherein at least a subset of the plurality of cells are intact and inviable.

77. The method of embodiment 76, wherein at least 50%, at least 60%, or at least 70% of the plurality of cells are intact after 1, 2, 3, 4, 5, 6, 7, 8, 9, or 10 days in the stabilization buffer at room temperature.

78. The method of embodiment 76, wherein the stabilization buffer maintains the size of the cells in the plurality within a range of 0.5 to 1× as compared with the size of the cells prior to contact with the stabilization buffer.

79. The method of embodiment 78, wherein the stabilization buffer maintains the size of the cells in the plurality within a range of ±20%, ±10%, or ±5% as compared with the size of the cells prior to contact with the stabilization buffer.

80. The method of embodiment 76, wherein the plurality of cells comprises at least two of a Hofbauer cell, a cytotrophoblast, a syncytiotrophoblast, an extravillous trophoblast, a villous core stroma, or a fetal vascular cell.

81. The composition of embodiment 76, wherein the plurality of cells comprises at least one of a cytotrophoblast or a syncytiotrophoblast.

82. The method of embodiment 76, wherein the cellulosic collection device is in or near the vagina for at least 10, 20, 30, 40, 50, 60, 90, 120, 150, or 180 minutes.

83. The method of embodiment 76, further comprising contacting the plurality of cells with a binding agent that specifically binds to a cell surface marker.

84. The method of embodiment 83, wherein the cell surface marker is a fetal cell surface marker.

85. The method of embodiment 83, wherein the cell surface marker is a maternal cell surface marker.

86. The method of embodiment 83, wherein after the contacting, the plurality of cells is enriched in one or more fetal cell types as compared to the plurality of cells before the contacting.

87. The method of embodiment 83, wherein the cell surface marker comprises FSHR, CD45, LHCGR, CD270, Trop2, FOLR1, CD114 (CSF3R) or SLC40A1.

88. The method of embodiment 83, wherein the binding agent is an antibody or fragment thereof.

89. The method of embodiment 83, wherein the binding agent comprises a magnetic bead.

90. The method of embodiment 83, wherein the binding agent comprises a detectable label.

91. The method of embodiment 83, further comprising isolating a subset of the plurality of cells that are bound to the binding agent.

92. The method of embodiment 83, wherein the plurality of cells comprises at least 500 cells.

93. The method of embodiment 83, wherein the plurality of cells comprises at least 0.5%, 1%, 2%, 5%, 10%, 20%, 25%, 30%, 40% or 50% trophoblast and trophoblast-derived cells.

94. The method of embodiment 83, wherein the population of cells is enriched for trophoblast and trophoblast-derived cells between about 2-fold and 2000-fold.

95. The method of embodiment 83, wherein the population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold.

96. The method of embodiment 76, further comprising quantifying a fetal fraction of the plurality of cells.

97. The method of embodiment 96, wherein quantifying a fetal fraction comprises intracellular staining, RNA analysis, short tandem repeat (STR) analysis, single nucleotide polymorphism (SNP) analysis, Y-chromosome analysis, or combinations thereof.

98. The method of embodiment 76, wherein the stabilization buffer comprises phosphate buffered saline, ethylenediaminetetraacetic acid, fetal bovine serum, Hank's balanced salt solution, collagenase, accutase, or a nuclease.

99. The method of embodiment 76, wherein the cellulosic collection device is a tampon, a pad, a plug, or a swab.

100. The method of embodiment 76, further comprising removing mucus from the plurality of cells.

101. The method of embodiment 76, further comprising analyzing at least one of a nucleic acid, a protein, a carbohydrate, or a lipid from the plurality of cells.

102. The method of embodiment 101, further comprising recommending a treatment to the pregnant subject.

103. A method for preserving a population of cells, the method comprising; (a) obtaining a vaginal fluid sample comprising a plurality of cells comprising at least one of a Hofbauer cell, a cytotrophoblast, a syncytiotrophoblast, an extravillous trophoblast, a villous core stroma, or a fetal vascular cell from a pregnant subject; and (b) placing the vaginal fluid sample in a stabilization buffer to create a mixture, wherein at least about 50% of the cells in the mixture are intact and inviable.

104. The method of embodiment 103, wherein at least 50%, at least 60%, or at least 70% of the cells in the mixture are intact after 1, 2, 3, 4, 5, 6, 7, 8, 9, or 10 days in the stabilization buffer at room temperature.

105. The method of embodiment 103, wherein the stabilization buffer maintains the size of the cells in the mixture within a range of 0.5 to 1× as compared with the size of the cells prior to contact with the stabilization buffer.

106. The method of embodiment 105, wherein the stabilization buffer maintains the size of the cells in the mixture within a range of ±20%, ±10%, or ±5% as compared with the size of the cells prior to contact with the stabilization buffer.

107. The method of embodiment 103, wherein the plurality of cells comprises at least two of a Hofbauer cell, a cytotrophoblast, a syncytiotrophoblast, an extravillous trophoblast, a villous core stroma, or a fetal vascular cell.

108. The composition of embodiment 103, wherein the plurality of cells comprises at least one of a cytotrophoblast or a syncytiotrophoblast.

109. The method of embodiment 103, further comprising contacting the cells in the mixture with a binding agent that specifically binds to a cell surface marker.

110. The method of embodiment 109, wherein the cell surface marker is a fetal cell surface marker.

111. The method of embodiment 109, wherein the cell surface marker is a maternal cell surface marker.

112. The method of embodiment 103, further comprising isolating an enriched population of cells by contacting the cells in the mixture with one or more binding agents that specifically bind to a cell surface marker, and separating at least a subset of the cells bound by the one or more binding agents to thereby form the enriched population.

113. The method of embodiment 112, wherein the cell surface marker is a maternal cell surface marker and the enriched population is depleted of maternal cells.

114. The method of embodiment 112, wherein the cell surface marker is a fetal cell surface marker and the enriched population is enriched in placenta derived fetal cells.

115. The method of embodiment 112, wherein the cell surface marker is a fetal cell surface marker and the enriched population is enriched in fetal trophoblast cells.

116. The method of embodiment 114, further comprising quantifying a fetal fraction of the plurality of cells.

117. The method of embodiment 115, wherein quantifying a fetal fraction comprises intracellular staining, RNA analysis, short tandem repeat (STR) analysis, single nucleotide polymorphism (SNP) analysis, Y-chromosome analysis, or combinations thereof.

118. The method of embodiment 114, wherein the enriched population is enriched in cytotrophoblasts, syncytiotrophoblasts, extravillous trophoblasts, or any combination thereof.

119. The method of any one of embodiments 109 to 118, wherein the cell surface marker comprises FSHR, CD45, LHCGR, CD270, Trop2, FOLR1, CD114 (CSF3R) or SLC40A1.

120. The method of any one of embodiments 109 to 119, wherein the binding agent is an antibody or fragment thereof.

121. The method of any one of embodiments 109 to 120, wherein the binding agent comprises a magnetic bead.

122. The method of any one of embodiments 109 to 121, wherein the binding agent comprises a detectable label.

123. The method of embodiment 103, wherein the population of cells comprises at least 500 cells.

124. The method of embodiment 103, wherein the population of cells comprises between 0.5% to 50% trophoblast and trophoblast-derived cells and at least 50%, at least 60%, or at least 70% of the enriched population are intact cells.

125. The method of embodiment 103, wherein the population of cells comprises at least 0.5%, 1%, 2%, 5%, 10%, 20%, 25%, 30%, 40% or 50% trophoblast and trophoblast-derived cells.

126. The method of embodiment 103, wherein the population of cells is enriched for trophoblast and trophoblast-derived cells between about 2-fold and 2000-fold.

127. The method of embodiment 103, wherein the population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold.

128. The method of embodiment 103, wherein the stabilization buffer comprises phosphate buffered saline, ethylenediaminetetraacetic acid, fetal bovine serum, Hank's balanced salt solution, collagenase, accutase, or a nuclease.

129. The method of embodiment 103, wherein the vaginal fluid sample is obtained with a cellulosic device comprising a tampon, a pad, a plug, or a swab.

130. The method of embodiment 129, wherein the device is placed in a vagina for at least 10, 20, 30, 40, 50, 60, 90, 120, 150, 180, 240, 300, 360, 420, or 480 minutes.

131. The method of embodiment 103, further comprising removing mucus from the population of cells.

132. The method of embodiment 103, further comprising analyzing at least one of a nucleic acid, a protein, a carbohydrate, or a lipid from the population of cells.

133. The method of embodiment 132, further comprising recommending a treatment to the pregnant subject.

134. A method for isolating a population of fetal trophoblast cells from a pregnant subject, the method comprising: (a) obtaining a mixture comprising (i) a vaginal fluid sample comprising a plurality of cells comprising at least one of a Hofbauer cell, a cytotrophoblast, a syncytiotrophoblast, an extravillous trophoblast, a villous core stroma, or a fetal vascular cell from the pregnant subject, and (ii) a stabilization buffer, wherein at least a subset of the plurality of cells are intact and inviable; (b) contacting the plurality of cells with at least one binding agent that specifically binds to a fetal cell surface protein; (c) isolating cells bound to the binding agent, thereby isolating the population of fetal trophoblast cells.

135. The method of embodiment 134, further comprising quantifying a fetal fraction of the plurality of cells.

136. The method of embodiment 135, wherein quantifying a fetal fraction comprises intracellular staining, RNA analysis, short tandem repeat (STR) analysis, single nucleotide polymorphism (SNP) analysis, Y-chromosome analysis, or combinations thereof.

137. The method of embodiment 134, wherein the cell surface marker comprises Trop2, FOLR1, CD114 (CSF3R) or SLC40A1.

138. The method of embodiment 134, further comprising contacting the plurality of cells with at least one binding agent that specifically binds to a maternal cell surface protein; and isolating cells bound to the binding agent, thereby isolating a population of maternal cells.

139. The method of embodiment 139, wherein the maternal cell surface protein comprises FSHR, CD45, LHCGR, or CD270.

140. The method of any one of embodiments 134 to 140, wherein the binding agent is an antibody or fragment thereof.

141. The method of any one of embodiments 134 to 141, wherein the binding agent comprises a magnetic bead.

142. The method of any one of embodiments 134 to 141, wherein the binding agent comprises a detectable label.

143. The method of embodiment 134, wherein the population of cells comprises at least two of a Hofbauer cell, a cytotrophoblast, a syncytiotrophoblast, an extravillous trophoblast, a villous core stroma, or a fetal vascular cell.

144. The composition of embodiment 134, wherein the population of cells comprises at least one of a cytotrophoblast or a syncytiotrophoblast.

145. The method of embodiment 134, wherein at least 50%, at least 60%, or at least 70% of the population of cells are intact after 1, 2, 3, 4, 5, 6, 7, 8, 9, or 10 days in the stabilization buffer at room temperature.

146. The method of embodiment 134, wherein the stabilization buffer maintains the size of the cells in the population within a range of 0.5 to 1× as compared with the size of the cells prior to contact with the stabilization buffer.

147. The method of embodiment 146, wherein the stabilization buffer maintains the size of the cells in the population within a range of ±20%, ±10%, or ±5% as compared with the size of the cells prior to contact with the stabilization buffer.

148. The method of embodiment 134, wherein the stabilization buffer comprises phosphate buffered saline, ethylenediaminetetraacetic acid, fetal bovine serum, Hank's balanced salt solution, collagenase, accutase, or a nuclease.

149. The method of embodiment 134, wherein the population of cells comprises at least 500 cells.

150. The method of embodiment 134, wherein the population of cells comprises at least 50% trophoblasts.

151. The method of embodiment 134, wherein the vaginal fluid sample is obtained with a cellulosic device comprising a tampon, a pad, a plug, or a swab.

152. The method of embodiment 151, wherein the cellulosic device is placed in a vagina for at least 10, 20, 30, 40, 50, 60, 90, 120, 150, or 180 minutes.

153. The method of embodiment 134, further comprising removing mucus from the population of cells.

154. The method of embodiment 134, further comprising analyzing at least one of a nucleic acid, a protein, a carbohydrate, or a lipid from the population of cells.

155. The method of embodiment 154, further comprising recommending a treatment to the pregnant subject.

156. A method for fetal cell analysis, comprising: (a) isolating a population of fetal trophoblast or placental derived cells comprising at least one of a Hofbauer cell, a cytotrophoblast, a syncytiotrophoblast, an extravillous trophoblast, a villous core stroma, or a fetal vascular cell from a vaginal fluid sample from a pregnant subject wherein at least 50% of the population of cells are intact and inviable; and (b) obtaining fetal nucleic acids from the population; and (c) analyzing at least one feature of the fetal nucleic acids.

157. The method of embodiment 156, further comprising quantifying a fetal fraction of the plurality of cells.

158. The method of embodiment 157, wherein quantifying a fetal fraction comprises intracellular staining, RNA analysis, short tandem repeat (STR) analysis, single nucleotide polymorphism (SNP) analysis, Y-chromosome analysis, or combinations thereof.

159. The method of embodiment 156, wherein the population of cells is enriched in at least two of a Hofbauer cell, a cytotrophoblast, a syncytiotrophoblast, an extravillous trophoblast, a villous core stroma, or a fetal vascular cell.

160. The composition of embodiment 156, wherein the population of cells comprises at least one of a cytotrophoblast or a syncytiotrophoblast.

161. The method of embodiment 156, further comprising, prior to (b), contacting the plurality of cells with at least one binding agent that specifically binds to a fetal cell surface protein.

162. The method of embodiment 156, wherein the cell surface marker comprises Trop2, FOLR1, CD114 (CSF3R) or SLC40A1.

163. The method of embodiment 156, further comprising contacting the plurality of cells with at least one binding agent that specifically binds to a maternal cell surface protein; and isolating cells bound to the binding agent, thereby isolating a population of maternal cells.

164. The method of embodiment 163, wherein the maternal cell surface protein comprises FSHR, CD45, LHCGR, or CD270.

165. The method of any one of embodiments 156 to 164, wherein the binding agent is an antibody or fragment thereof.

166. The method of any one of embodiments 156 to 165, wherein the binding agent comprises a magnetic bead.

167. The method of any one of embodiments 156 to 165, wherein the binding agent comprises a detectable label.

168. The method of embodiment 156, wherein the feature comprises a copy number variant, a single nucleotide variant, an insertion, a deletion, an epigenetic modification, a post-translational protein modification, or a combination thereof.

169. The method of embodiment 168, wherein the epigenetic modification comprises methylation, phosphorylation, ubiquitination, sumoylation, acetylation, ribosylation, citrullination, or a combination thereof.

170. The method of embodiment 156, further comprising labeling at least one of the nucleic acids prior to analysis.

171. The method of embodiment 156, further comprising contacting the vaginal fluid sample to a stabilization buffer.

172. The method of embodiment 171, wherein at least 50%, at least 60%, or at least 70% of the population of cells are intact after 1, 2, 3, 4, 5, 6, 7, 8, 9, or 10 days in the stabilization buffer at room temperature.

173. The method of embodiment 171, wherein the stabilization buffer maintains the size of the cells in the population within a range of 0.5 to 1× as compared with the size of the cells prior to contact with the stabilization buffer.

174. The method of embodiment 173, wherein the stabilization buffer maintains the size of the cells in the population within a range of ±20%, ±10%, or ±5% as compared with the size of the cells prior to contact with the stabilization buffer.

175. The method of embodiment 171, wherein the stabilization buffer comprises phosphate buffered saline, ethylenediaminetetraacetic acid, fetal bovine serum, Hank's balanced salt solution, collagenase, accutase, or a nuclease.

176. The method of embodiment 156, wherein the population of cells comprises at least 500 cells.

177. The method of embodiment 156, wherein the population of cells comprises at least 50% trophoblasts.

178. The method of embodiment 156, wherein the vaginal fluid sample is obtained with a cellulosic device comprising a tampon, a pad, a plug, or a swab.

179. The method of embodiment 178, wherein the device is placed in a vagina for at least 10, 20, 30, 40, 50, 60, 90, 120, 150, or 180 minutes.

180. The method of embodiment 156, further comprising removing mucus from the population of cells.

181. The method of embodiment 156, further comprising analyzing at least one of a protein, a carbohydrate, or a lipid from the population of cells.

182. The method of embodiment 181, further comprising recommending a treatment to the pregnant subject.

183. The method of embodiment 156, wherein the analysis comprises one or more of PCR, sequencing, karyotyping, in situ hybridization, immunofluorescence, FACS, or mass spectroscopy.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Collection of Cervicovaginal Samples from Pregnant Subjects

Nineteen cervicovaginal samples from pregnant women were collected in preservation buffer. RNA was extracted from total cell lysate and qPCR was performed using fetal specific HLA-g and housekeeping gene 18s. PSA and SRY expression was also measured on the DNA from these samples in order to determine semen contamination and sex of fetus. If PSA was positive, then sex of fetus could not be determined. All HLA-g expression was normalized to 18s. All samples produced a very high expression of HLA-g compared to non-pregnant control sample except for TB09 which was lost in the mail and had highly degraded material. This likely also resulted in the very low HLA-g expression in this sample.

TABLE 1
PCR Analysis of Cervicovaginal Samples

| | | | | HLAg | | | | | | | SRY |
|---|---|---|---|---|---|---|---|---|---|---|---|
| | 18s | | | mean | | | [C] | Total | HLA | PSA | nested |
| | mean | 18s | 18s | copies/ | HLAg | HLA | DNA | DNA | PCR | PCR | PCR |
| Sample | ng/ul | StDev | norm | ul | StDev | norm | ng/ul | ug | RNA | RNA | DNA |
| Non- | 4.41E+00 | 9.22E−01 | 3.89E+00 | 7.30E+01 | 4.78E+01 | 1.88E+01 | NA | NA | NA | NA | NA |
| pregnant | | | | | | | | | | | |
| Negative | 5.28E−05 | 1.31E−05 | 4.65E−05 | 0.00E+00 | 0.00E+00 | 0.00E+00 | NA | NA | NA | NA | NA |
| TB01 | 3.52E+00 | 7.85E−01 | 3.10E+00 | 3.39E+03 | 2.29E+03 | 1.09E+03 | 9.8 | 2.94 | Positive | Positive | Cannot |
| | | | | | | | | | | | be deter- |
| | | | | | | | | | | | mined |
| TB03 | 3.56E+00 | 1.12E+00 | 3.14E+00 | 5.61E+03 | 9.28E+02 | 1.79E+03 | 35.3 | 21.18 | Positive | Negative | Negative |
| TB04 | 2.84E+00 | 6.39E−01 | 2.50E+00 | 1.42E+04 | 4.95E+03 | 5.68E+03 | 10.9 | 6.54 | Positive | Negative | Negative |
| TB05 | 7.48E−01 | 2.70E−01 | 6.59E−01 | 7.74E+03 | 1.70E+03 | 1.17E+04 | 85.6 | 51.36 | Positive | Negative | Positive |
| TB06 | 1.76E+01 | 3.69E+00 | 1.55E+01 | 2.99E+05 | 6.25E+04 | 1.93E+04 | 68 | 40.8 | Positive | Negative | Positive |
| TB07 | 5.99E+01 | 2.88E+01 | 5.27E+01 | 3.06E+05 | 1.17E+05 | 5.80E+03 | 36.6 | 10.98 | Positive | Positive | Cannot |
| | | | | | | | | | | | be deter- |
| | | | | | | | | | | | mined |
| TB08 | 1.70E+00 | 7.36E−01 | 1.50E+00 | 1.64E+04 | 5.28E+03 | 1.09E+04 | 46.3 | 13.89 | Positive | Negative | Positive |
| TB09 | 3.64E+01 | 2.84E+00 | 3.20E+01 | 4.68E+00 | 4.44E+00 | 1.46E−01 | 32.3 | 19.38 | Negative | Positive | Cannot |
| | | | | | | | | | | | be deter- |
| | | | | | | | | | | | mined |
| TB10 | 1.47E+00 | 6.13E−01 | 1.30E+00 | 7.56E+04 | 2.10E+04 | 5.83E+04 | 14.1 | 8.46 | Positive | Negative | Negative |
| TB11 | 1.50E+00 | 3.12E−01 | 1.32E+00 | 1.99E+04 | 7.91E+03 | 1.50E+04 | 80.4 | 24.12 | Positive | Negative | Negative |
| TB12 | 8.44E+01 | 1.78E+01 | 7.43E+01 | 3.02E+05 | 8.11E+04 | 4.07E+03 | 57 | 17.1 | Positive | Negative | Negative |
| TB13 | 3.63E+00 | 2.98E+00 | 3.19E+00 | 1.44E+05 | 8.56E+04 | 4.52E+04 | 43 | 12.9 | Positive | Positive | Cannot |
| | | | | | | | | | | | be deter- |
| | | | | | | | | | | | mined |
| TB14 | 1.14E+00 | 3.25E−01 | 1.00E+00 | 5.10E+03 | 4.50E+02 | 5.10E+03 | 32.1 | 9.63 | Positive | Negative | Positive |
| TB15 | 2.68E+01 | 4.10E+00 | 2.36E+01 | 2.62E+05 | 1.16E+05 | 1.11E+04 | 29.2 | 5.84 | Positive | Negative | Negative |
| TB16 | 1.69E−01 | 3.14E−02 | 1.49E−01 | 4.74E+02 | 8.40E+01 | 3.18E+03 | 9.94 | 1.988 | Positive | Negative | Negative |
| TB18 | 4.46E+01 | 6.52E+00 | 3.93E+01 | 6.26E+05 | 2.52E+05 | 1.59E+04 | 27.2 | 5.44 | Positive | Negative | Negative |
| TB19 | 9.87E+00 | 2.24E+00 | 8.69E+00 | 1.62E+05 | 7.69E+04 | 1.87E+04 | 10.3 | 2.06 | Positive | Positive | Cannot |
| | | | | | | | | | | | be deter- |
| | | | | | | | | | | | mined |
| TB20 | 7.27E+00 | 7.33E−01 | 6.40E+00 | 2.81E+05 | 1.86E+05 | 4.40E+04 | 7.16 | 1.432 | Positive | Negative | Negative |
| TB23 | 1.14E+01 | 1.22E+00 | 1.00E+01 | 6.37E+05 | 4.44E+05 | 6.35E+04 | 7.62 | 1.524 | Positive | Negative | Negative |

HLA-g expression was orders of magnitude greater in pregnant samples than in non-pregnant controls. It is then concluded that fetal trophoblast cells are present in sufficient quantities in cervicovaginal samples from pregnant women to warrant attempts to isolate fetal trophoblast cells from this sample matrix.

### Example 2: Antibodies for Enrichment of Maternal or Fetal Trophoblast Cells

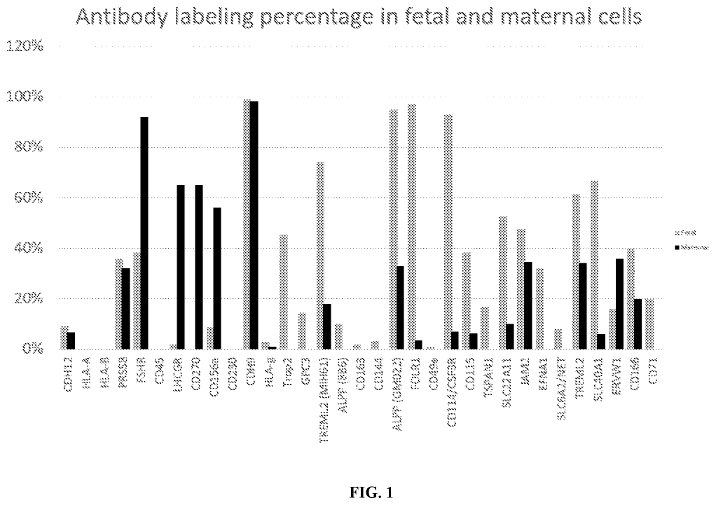

Both RNA expression analysis, through sequencing, and antibody screen were performed to understand cell surface expression differences between maternal and fetal trophoblast cells. For sequencing experiments, fetal HTR8 trophoblast cells were spiked into non-pregnant cervicovaginal samples to identify genes with a large fold increase in fetal to maternal cells. Strong candidates were curated, along with promising candidates from literature, and antibodies for these genes were used for antibody screening. Antibodies were screened against non-pregnant cervicovaginal samples as well as fetal JAR trophoblasts cells (male derived fetal trophoblasts). Percent staining was recorded, as well as primary cell types that express these antibodies, to understand the cell composition of the sample. These data are summarized in FIG. 1 and Tables 2-4.

TABLE 2
Candidates for Cell Surface Targets for
Maternal and Fetal Trophoblast Cells

| Antibody | |
|---|---|
| Target | Tissue Specificity |
| CDH12 | Glandular cells |
| HLA-A | Maternal decidua, glandular cells, epithelial cells, |
| | stromal cells |
| HLA-B | Maternal decidua, glandular cells, epithelial cells, |
| | stromal cells |
| PRSS8 | maternal decidua and trophoblast cells |
| FSHR | epithelial, endothelial, fibroblast, maternal decidua |
| CD45 | immune cells |
| LHCGR | glandular cells, epithelial, stromal cells, |
| CD270 | glandular cells, epithelial, maternal decidua |
| CD156a | glandular cells, epithelial, maternal and fetal decidua |
| CD230 | Neuronal cells, glial cells |
| CDH9 | epithelial, trophoblast |
| HLA-g | Extravillous trophoblasts |
| Trop2 | Syncytiotrophoblasts, cytotrophoblasts |
| GPC3 | Maternal decidua |
| TREML2 | syncytiotrophoblasts, cytotrophoblasts, myeloid cells |
| (MIH61) | (monocyte precursors) |
| ALPP (8B6) | glandular cells, epithelial, trophoblasts |
| CD163 | glandular cells, maternal decidua |
| CD144 | glandular cells, epithelial, stromal cells, |
| ALPP | syncytiotrophoblasts, cytotrophoblasts, myeloid cells |
| (GM022) | (monocyte precursors) |
| FOLR1 | syncytotrophoblasts |
| CD49e | trophoblasts, maternal decidua |
| CD114/CSF3R | extravillous trophoblasts, Hofbauer cells, some immune |
| | cells, endothelial |
| CD115 | glandular cells, epithelial, maternal and fetal decidua |
| TSPAN1 | glandular cells |
| SLC22A11 | extravillous trophoblasts, glandular cells, epithelial |
| JAM2 | extravillous trophoblasts, glandular cells, epithelial |
| EFNA1 | glandular cell, endothelial, epithelial, stromal, placental |
| SLC6A2/NET | fetal endothelia |
| TREML2 | syncytiotrophoblasts, cytotrophoblasts, myeloid cells |
| | (monocyte precursors) |
| SLC40A1 | syncytiotrophoblasts |
| ERVW1 | cytotrophoblasts, epithelial, glandular cells |
| CD166 | glandular cells, epithelial, maternal and fetal decidua |
| CD71 | glandular cells, epithelial, macrophages, decidua, |
| | trophoblasts |

TABLE 3
Maternal and Fetal Expression

| | Mean Maternal | Mean Fetal | Fold |
|---|---|---|---|
| Antibody | RNA | RNA | Change |
| Target | Expression | Expression | (log10) |
| CDH12 | 3.0 | 0.0 | −3.0 |
| HLA-A | Not Detected | Not Detected | Not Detected |
| HLA-B | 1.1 | 0.0 | 1.2 |
| PRSS8 | 2.7 | 0.1 | −2.6 |
| FSHR | 2.5 | 0.0 | −2.4 |
| CD45 | Not Detected | Not Detected | Not Detected |
| LHCGR | 2.6 | 0.1 | −2.6 |
| CD270 | 0.0 | 0.1 | 0.1 |
| CD156a | 2.0 | 0.3 | −1.6 |
| CD230 | 2.0 | 2.8 | 0.8 |
| CDH9 | 2.8 | 0.0 | −2.8 |
| HLA-g | 2.0 | 0.0 | −2.0 |
| Trop2 | Not Detected | Not Detected | Not Detected |
| GPC3 | 1.6 | 0.0 | −1.6 |
| TREML2 | 1.7 | 0.0 | −1.7 |
| (MIH61) | | | |
| ALPP (8B6) | 0.0 | 0.1 | 0.1 |
| CD163 | 0.1 | 0.0 | −0.1 |
| CD144 | 2.0 | 0.0 | −2.0 |
| ALPP (GM022) | 0.0 | 0.1 | 0.1 |
| FOLR1 | Not Detected | Not Detected | Not Detected |
| CD49e | Not Detected | Not Detected | Not Detected |
| CD114/CSF3R | 1.7 | 0.0 | −1.6 |
| CD115 | 0.0 | 0.0 | 0.0 |
| TSPAN1 | 1.3 | 0.4 | −0.9 |
| SLC22A11 | 0.0 | 0.0 | 0.0 |
| JAM2 | Not Detected | Not Detected | Not Detected |
| EFNA1 | 0.1 | 0.6 | 0.5 |
| SLC6A2/NET | Not Detected | Not Detected | Not Detected |
| TREML2 | 1.7 | 0.0 | −1.7 |
| SLC40A1 | Not Detected | Not Detected | Not Detected |
| ERVW1 | 2.3 | 0.0 | −2.2 |
| CD166 | 0.1 | 2.4 | 2.3 |
| CD71 | Not Detected | Not Detected | Not Detected |

TABLE 4
Maternal and Fetal Expression

| | FETAL | MATER- | |
|---|---|---|---|
| | SPECI- | NAL SPEC- | |
| | FICITY | IFICITY | |
| | (% of fetal | (% of mater- | |
| Antibody | trophoblast | nal cells | |
| Target | stained) | stained) | Note |
| CDH12 | 9% | 7% | |
| HLA-A | 0% | 1-10% | |
| HLA-B | 0% | 1-10% | |
| PRSS8 | 36% | 32% | |
| FSHR | 38% | 92% | Novel maternal found in |
| | | | gene expression and |
| | | | antibody screen |
| CD45 | 0% | 42-62% | |
| LHCGR | 2% | 65% | |
| CD270 | 0% | 65% | Novel maternal found in |
| | | | gene expression and |
| | | | antibody screen |
| CD156a | 9% | 56% | Novel maternal found in |
| | | | gene expression and |
| | | | antibody screen |
| CD230 | 0% | 0% | |
| CDH9 | 99% | 98% | |
| HLA-g | 3% | 1% | |
| Trop2 | 45% | 0% | |
| GPC3 | 14% | NA | |
| TREML2 | 74% | 18% | |
| (MIH61) | | | |
| ALPP (8B6) | 10% | NA | |
| CD163 | 2% | low | |
| CD144 | 3% | 0 | |
| ALPP | 95% | 33% | |
| (GM022) | | | |
| FOLR1 | 97% | 3.60% | Novel fetal found in lit |
| | | | review but for cancer - |
| | | | NGJ hypothesis that fetal |
| | | | trophoblast cells express |
| | | | a lot of folate receptors. |
| | | | Confirmed through antibody |
| | | | screen |
| CD49e | 1% | NA | |
| CD114/ | 93% | 7% | Novel through gene |
| CSF3R | | | expression and antibody |
| | | | screen |
| CD115 | 38% | 6.30% | |
| TSPAN1 | 17% | NA | |
| SLC22A11 | 53% | 10% | |
| JAM2 | 48% | 34.60% | |
| EFNA1 | 32% | NA | |
| SLC6A2/ | 8% | NA | |
| NET | | | |
| TREML2 | 62% | 34.30% | |
| SLC40A1 | 67% | 5.92% | |
| ERVW1 | 16% | 36% | |
| CD166 | 40% | 20% | |
| CD71 | 20% | NA | |

These data show that RNA expression and antibody staining did not totally match up. This could be due to the fact that different fetal cell lines were used for analysis, however, many antibodies do correlate, and may be a good method for simply canvasing the diversity represented in fetal trophoblast cells. There is no single antibody that distinguishes the cells, and therefore a combination of antibodies will be used. This will allow for a belier characterization of cell diversity.

### Example 3: Stabilization Buffer Testing

Several commercially available zwitterionic stabilization buffers were identified for testing. Between 3-6 million HeLa cervical cells were added into each buffer according to the manufacturers recommended dilution in either water or phosphate buffered saline. The number of cells for input was quantified before addition of buffer. A cutoff of at least 50% intact cells was used as a threshold to move buffer to round 2 of testing. Mawi, Norgen cf-DNA, and PaxGene buffers passed this initial round of testing. Cells were quantified on the MoxiZ cell counter.

TABLE 5
Buffer Testing Round 1

| | | Percentage of intact | | |
|---|---|---|---|---|
| | | Cells | | |
| Company | Product | Day 1 | Day 6 | Comments |
| MagBio | Blood STASIS 21-cfDNA | >50% | <50% | |
| | Blood Collection Tubes | | | |
| MagBio | Blood STASIS-CTC Blood | >50% | <50% | |
| | Collection Tubes | | | |
| PaxGene | PAXgene Blood ccfDNA | >50% | >50% | Used for round |
| | | | | 2 testing |
| Cytomark | TransFix | >50% | <50% | |
| Mawi | iSWAB Cells | >50% | >50% | Used for round |
| | | | | 2 testing |
| Biomatrica | LBgard | >50% | <50% | |
| Norgen | cf-DNA/cf-RNA | >50% | >50% | Used for round |
| | Preservative | | | 2 testing |
| Norgen | Urine Preservation | >50% | <50% | |
| Streck | Industry Standard | TBD | TBD | |

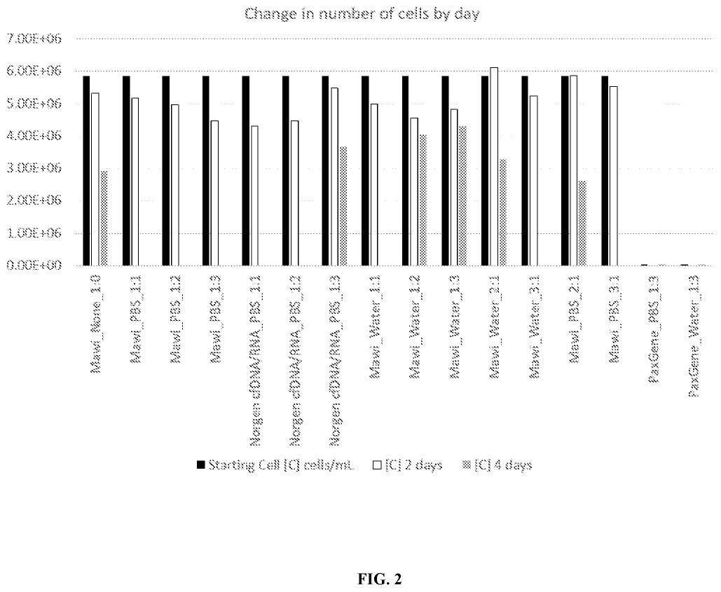

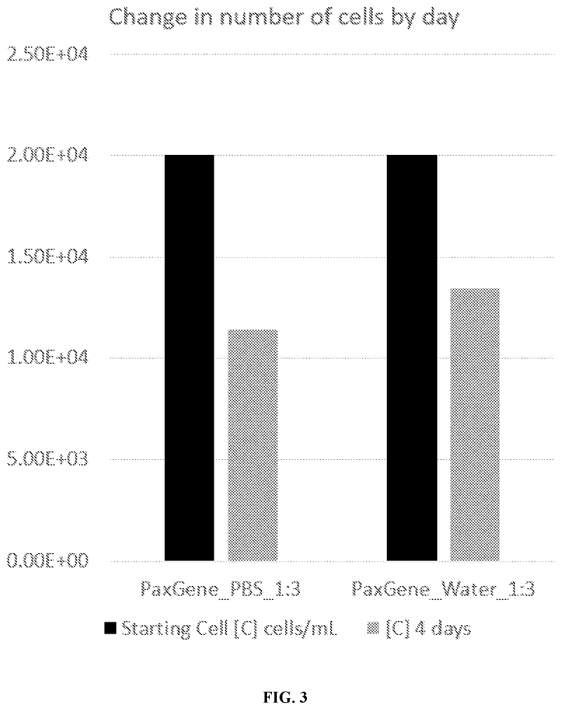

Buffers that passed round 1 testing were tested at different dilutions of either water or phosphate buffered saline and intact PBMC cells were quantified at day 2 and day 4 post buffer addition. Buffers that have at least 60% intact cells by day 4, and had no other concerning characteristics, were subjected to a 3rd round of testing. Size of cells was also noted in this experiment. Cells were quantified on the MoxiZ cell counter. The only two buffers that passed this test were Norgen cf-DNA/cf-RNA buffer and Paxgene buffer. While Mawi buffer had good recovery at day 4 for 2:1 and 3:1 dilutions in water, the osmolality of the buffer dilutions were too low, causing the cells to enlarge, potentially impacting the long-term integrity of the cell membrane. These data are summarized in FIG. 2 and FIG. 3 and Tables 6-7.

TABLE 6
Buffer Testing Round 2

| | | | | Starting | Original | | 2 days | | 2 days |
|---|---|---|---|---|---|---|---|---|---|
| | | | | Cell [C] | Size | [C] | delta | Size | delta |
| Buffer | Conditions | Diluent | Dilution | (cells/ml) | (uM) | 2 days | [C] | 2 day | size |
| Mawi | Mawi_None_1:0 | None | 1:0 | 5.87E+06 | 11.849 | 5.32E+06 | 90.63% | 10.965 | 92.54% |
| Mawi | Mawi_PBS_1:1 | PBS | 1:1 | 5.87E+06 | 11.849 | 5.16E+06 | 87.90% | 10.444 | 88.14% |
| Mawi | Mawi_PBS_1:2 | PBS | 1:2 | 5.87E+06 | 11.849 | 4.97E+06 | 84.67% | 10.152 | 85.68% |
| Mawi | Mawi_PBS_1:3 | PBS | 1:3 | 5.87E+06 | 11.849 | 4.47E+06 | 76.15% | 10.32 | 87.10% |
| Norgen | Norgen | PBS | 1:1 | 5.87E+06 | 11.849 | 4.32E+06 | 73.59% | 8.939 | 75.44% |
| cfDNA/ | cfDNA/RNA_ | | | | | | | | |
| RNA | PBS_1:1 | | | | | | | | |
| Norgen | Norgen | PBS | 1:2 | 5.87E+06 | 11.849 | 4.46E+06 | 75.98% | 8.547 | 72.13% |
| cfDNA/ | cfDNA/RNA_ | | | | | | | | |
| RNA | PBS_1:2 | | | | | | | | |
| Norgen | Norgen | PBS | 1:3 | 5.87E+06 | 11.849 | 5.48E+06 | 93.36% | 8.32 | 70.22% |
| cfDNA/ | cfDNA/RNA_ | | | | | | | | |
| RNA | PBS_1:3 | | | | | | | | |
| Mawi | Mawi_Water_1:1 | Water | 1:1 | 5.87E+06 | 11.849 | 5.00E+06 | 85.18% | 11.043 | 93.20% |
| Mawi | Mawi_Water_1:2 | Water | 1:2 | 5.87E+06 | 11.849 | 4.55E+06 | 77.51% | 12.143 | 102.48% |
| Mawi | Mawi_Water_1:3 | Water | 1:3 | 5.87E+06 | 11.849 | 4.83E+06 | 82.28% | 12.231 | 103.22% |
| Mawi | Mawi_Water_2:1 | Water | 2:1 | 5.87E+06 | 11.849 | 6.12E+06 | 104.26% | 10.907 | 92.05% |
| Mawi | Mawi_Water_3:1 | Water | 3:1 | 5.87E+06 | 11.849 | 5.24E+06 | 89.27% | 10.885 | 91.86% |
| Mawi | Mawi_PBS_2:1 | PBS | 2:1 | 5.87E+06 | 11.849 | 5.86E+06 | 99.83% | 10.479 | 88.44% |
| Mawi | Mawi_PBS_3:1 | PBS | 3:1 | 5.87E+06 | 11.849 | 5.52E+06 | 94.04% | 10.511 | 88.71% |
| Pax | PaxGene_PBS_1:3 | PBS | 1:3 | 2.00E+04 | 11.849 | Not | NA | Not | NA |
| Gene | | | | | | Measured | | Measured | |
| Pax | PaxGene_Water_1:3 | Water | 1:3 | 2.00E+04 | 11.849 | Not | NA | Not | NA |
| Gene | | | | | | Measured | | Measured | |

TABLE 7
Buffer Testing Round 2

| | | | | | 4 days | | 4 days | |
|---|---|---|---|---|---|---|---|---|
| | | | | [C] | delta | Size | delta | |
| Buffer | Conditions | Diluent | Dilution | 4 days | [C] | 4 day | size | Comments |
| Mawi | Mawi_None_1:0 | None | 1:0 | 2.93E+06 | 49.91% | 7.423 | 62.65% | |
| Mawi | Mawi_PBS_1:1 | PBS | 1:1 | | | | | Cells clumped |
| | | | | | | | | and recovery |
| | | | | | | | | was low at day 4 |
| Mawi | Mawi_PBS_1:2 | PBS | 1:2 | | | | | Cells clumped |
| | | | | | | | | and recovery |
| | | | | | | | | was low at day 4 |
| Mawi | Mawi_PBS_1:3 | PBS | 1:3 | | | | | Cells clumped |
| | | | | | | | | and recovery |
| | | | | | | | | was low at day 4 |
| Norgen | Norgen | PBS | 1:1 | | | | | Cells clumped |
| cfDNA/ | cfDNA/RNA_ | | | | | | | and recovery |
| RNA | PBS_1:1 | | | | | | | was low at day 4 |
| Norgen | Norgen | PBS | 1:2 | | | | | Cells clumped |
| cfDNA/ | cfDNA/RNA_ | | | | | | | and recovery |
| RNA | PBS_1:2 | | | | | | | was low at day 4 |
| Norgen | Norgen | PBS | 1:3 | 3.66E+06 | 62.35% | 6.546 | 55.25% | Taken into |
| cfDNA/ | cfDNA/RNA_ | | | | | | | round 3 |
| RNA | PBS_1:3 | | | | | | | |
| Mawi | Mawi_Water_1:1 | Water | 1:1 | *** | | | | Cells clumped |
| | | | | | | | | and recovery |
| | | | | | | | | was low at day 4 |
| Mawi | Mawi_Water_1:2 | Water | 1:2 | 4.06E+06 | 69.17% | 11.266 | 95.08% | Cells enlarged. |
| | | | | | | | | Sample |
| | | | | | | | | processing |
| | | | | | | | | increased cell |
| | | | | | | | | degradation |
| Mawi | Mawi_Water_1:3 | Water | 1:3 | 4.29E+06 | 73.08% | 10.796 | 91.11% | Cells enlarged. |
| | | | | | | | | Sample |
| | | | | | | | | processing |
| | | | | | | | | increased cell |
| | | | | | | | | degradation |
| Mawi | Mawi_Water_2:1 | Water | 2:1 | 3.28E+06 | 55.88% | 7.431 | 62.71% | |
| Mawi | Mawi_Water_3:1 | Water | 3:1 | *** | | | | Cells clumped |
| | | | | | | | | and recovery |
| | | | | | | | | was low at day 4 |
| Mawi | Mawi_PBS_2:1 | PBS | 2:1 | 2.60E+06 | 44.29% | 6.731 | 56.81% | |
| Mawi | Mawi_PBS_3:1 | PBS | 3:1 | *** | | | | Cells clumped |
| | | | | | | | | and recovery |
| | | | | | | | | was low at day 4 |
| Pax | PaxGene_PBS_1:3 | PBS | 1:3 | 1.14E+04 | 57.60% | Not | NA | |
| Gene | | | | | | Measured | | |
| Pax | PaxGene_Water_1:3 | Water | 1:3 | 1.34E+04 | 66.90% | Not | NA | Taken into |
| Gene | | | | | | Measured | | round 3 |

*** Cell clumping post collection causes cells to adhere to matrix of cotton tampon, potentially reducing the overall recovery of cells, making these buffer combinations unsuitable for our tampon based collection method.

Both Norgen and Paxgene buffers were supplied as concentrates, and are typically diluted by addition of whole blood (products' commercial intent). Because of this, and since there is not extra fluid in cervical samples to act as a diluent, both water and PBS were tested to act as a diluent in our product.

Norgen and Paxgene provided the most robust cell count at day 4 without increasing size of cells or causing cells to adhere.

The purpose of the next experiment was to assess whether the stabilization buffers that passed the first two rounds of experiments had an effect on the cell surface markers that we use for trophoblast cell enrichments. A 50:50 mix of cervical cells from non-pregnant patients, and JAR trophoblast cells were incubated in both PaxGene and Norgen cf-DNA buffer, and after 6 days, the cells were stained with maternal and fetal antibodies in the selected antibody enrichment panel. Single stain controls were used for compensation, and fluorescent intensities were measured on the Guava EasyCyte flow cytometer. FCS files were imported into FlowJo to determine the number of positively labeled cells between the two buffers. Initially a 15% reduction in maternal antibody combination testing was observed, and so each maternal antibody was tested alone. Specifically CD270 shows almost a 20% reduction in staining cells in the PaxGene buffer compared to Norgen. The chemistry of the PaxGen buffer directly interferes with the cell surface markers of interest.

TABLE 8
Buffer Testing Round 3

| | | | Norgen | | |
|---|---|---|---|---|---|
| Antibody Measured | | PaxGene | cf-DNA | Delta in | |
| and Stain (top | | 1:3 in | 1:8 in | stained | |
| candidates) | Cell specificity | Water | PBS | cells | |
| LHCGR/CD270 - F1TC | Glandular | 30.60% | 45.70% | 15.10% | Reduction in |
| | cells/Epithelial | | | | staining for this |
| | | | | | maternal |
| | | | | | antibody |
| | | | | | combination by |
| | | | | | 15% |
| TROP2/FOLR1/ | Syncytiot-/cyto-/ | 41.90% | 44.10% | 2.20% | |
| CD114-PE | extravillous trophoblast | | | | |
| CD45 - APC | Immune cells | 54.70% | 47.80% | −6.90% | |
| CD270 - FITC | Glandular | 26.80% | 46.30% | 19.50% | Reduction in |
| | cells/Epithelial/maternal | | | | staining for this |
| | decidua | | | | maternal |
| | | | | | antibody by |
| | | | | | almost 20% |

Some buffers interact with cell surface markers preventing binding of target antibodies to the cell, reducing the overall recovery or depletion of cell types.

Next, differing concentrations of PBS were used to determine whether lower salt concentrations could help ameliorate the reduction in cell size that was observed in round 2 testing. Dilution of buffer to PBS was tested to determine recovery of cell size without significant loss of cells.

TABLE 9
Osmolality range on cell size

| | | | |
|---|---|---|---|
| 1X PBS | 260 | 300 | mOsm/kg H2O |
| 0.05X PBS | 130 | 150 | mOsm/kg H2O |
| H2O | 0 | 0 | mOsm/kg H2O |
| Undilute Norgen Buffer | Unknown | | mOsm/kg H2O |

TABLE 10
Buffer Testing Round 4

| Dilution of | Dilution of | % Change in cell |
|---|---|---|
| Norgen Buffer | PBS or H2O | size (from 22 um) |
| 1:1 | 1X PBS | −24.56% |
| 1:2 | 1X PBS | −27.87% |
| 1:3 | 1X PBS | −29.78% |
| 1:8 | 1X PBS | −19.09% |
| 1:8 | 0.05X PBS | −5.45% |
| 1:8 | H2O | NA Cell clumping makes it |
| | | difficult to assess size |

Maintaining cell size is important as a size selection is conducted during processing in order to optimize cell recovery. Reduction in cell size so dramatically can cause size discrimination problems with bacteria and other unwanted cell types. A lower PBS concentration enabled the best preservation of cell size.

### Example 4: Cell Dissociation Conditions

Next it was determined what reagents and temperature conditions work best for dissociating cells without affecting recovery of intact cells. Total number of cells recovered and preservation of the HLA-A/B surface marker were tested after dissociation. Changing salt composition, protein amount, protease activity of digestive enzymes can change how gentle of a dissociation can be in order to dissociate the most cells possible without harming cell surface markers. Cells were labeled with a PE labeled HLA-A/B antibody. DNase I has since been added to degrade any free floating DNA that may stick to the outside of the cell and cause maternal background noise after cell isolations.

TABLE 11
Dissociation Results 10 minutes at 37 degrees C.

| | Percentage | Total | | | |
|---|---|---|---|---|---|
| | PE+ | Cells | | | File |
| Unstained | 3.74% | 3907 | | | 62 |
| 1xPBS + 0.5% FBS + EDTA | 22.50% | 2325 | 523 | 1802 | 68 |
| PBS | 18.90% | 1528 | 289 | 1239 | 76 |
| HBSS | 18.70% | 2149 | 402 | 1747 | 77 |
| 1xPBS + 0.5% FBS + | 26.20% | 2932 | 768 | 2164 | 78 |
| EDTA + Collagenase 4 | | | | | |
| PBS + Collagenase 4 | 22.00% | 2658 | 585 | 2073 | 79 |
| HBSS + Collagenase 4 | 13.50% | 2052 | 277 | 1775 | 80 |
| Accutase | 14.00% | 2436 | 341 | 2095 | 81 |
| PBS + Accutase | 19.60% | 3001 | 588 | 2413 | 69 |

TABLE 12
Dissociation Results 30 minutes at 37 degrees C.

| | Percentage | Total | | | |
|---|---|---|---|---|---|
| | PE+ | Cells | | | File |
| Unstained | 3.74% | 3907 | | | 62 |
| 1xPBS + 0.5% FBS + EDTA | 28.70% | 2669 | 766 | 1903 | 70 |
| PBS | 18.00% | 2688 | 484 | 2204 | 84 |
| HBSS | 21.60% | 2732 | 590 | 2142 | 87 |
| 1xPBS + 0.5% FBS + | 24.60% | 2893 | 712 | 2181 | 89 |
| EDTA + Collagenase 4 | | | | | |
| PBS + Collagenase 4 | 10.90% | 2316 | 252 | 2064 | 74 |
| HBSS + Collagenase 4 | 18.50% | 2634 | 487 | 2147 | 85 |
| Accutase | 11.80% | 2707 | 319 | 2388 | 88 |
| PBS + Accutase | 13.10% | 2599 | 340 | 2259 | 75 |

From these results it has been found that there may be an optimal reagent and dissociation condition that are particularly suited to our specific cell composition.

### Example 5: Column-Based Magnetic Enrichment

Male JAR fetal trophoblasts were spiked into non-pregnant cervicovaginal cells at increasing amounts (similar to gene expression experiment), and maternal and fetal antibodies were used to enrich sample on Miltenyi MACS magnetic columns. RNaseP and the Y chromosome SRY copy number assays were used to quantify male fetal DNA to overall DNA present in the sample in order to get a fetal fraction estimate. Pre-enrichment analysis shows that Y chromosome is present at similar quantities to cell count spike ins and actual DNA composition. Two rounds of maternal depletion (CD45 then LHCGR/CD270) were performed. Then one round of fetal enrichment with CD114/HLA-g/FOLR1. Maternal material was depleted at an average of 100×, and fetal material was enriched at an average of 10×. Due to the heavy processing of the cells, about 70% of total material was lost in the processing.

TABLE 13
Pre-enrichment

| Sample | RNaseP Qty Mean | SRY Qty Mean | |
|---|---|---|---|
| 1P - 100% M | 8.87E+03 | 0.00E+00 | 0.00% |
| 2P - 0.05% F | 1.26E+04 | 8.13E+00 | 0.06% |
| 3P - 0.1% F | 7.69E+03 | 1.13E+01 | 0.15% |
| 4P - 1% F | 1.05E+04 | 1.04E+02 | 0.98% |
| 5P - 10% F | 1.04E+04 | 8.51E+02 | 8.18% |
| 6P - 100% F | 2.57E+03 | 2.69E+03 | 105.01% |

TABLE 14
Maternal Fraction

| Sample | RNaseP Qty Mean | SRY Qty Mean | |
|---|---|---|---|
| 1M - 100% M | 1.44E+04 | 0.00E+00 | 0.00% |
| 2M - 0.05% F | 1.48E+04 | 4.85E+00 | 0.03% |
| 3M - 0.1% F | 1.51E+04 | 1.02E+01 | 0.07% |
| 4M - 1% F | 1.06E+04 | 1.06E+02 | 1.00% |
| 5M - 10% F | 9.44E+03 | 1.03E+03 | 10.89% |
| 6M - 100% F | 1.95E+03 | 1.91E+03 | 98.14% |

TABLE 15
Fetal Fraction

| Sample | RNaseP Qty Mean | SRY Qty Mean | |
|---|---|---|---|
| 1F - 100% M | 3.98E+02 | 0.00E+00 | 0.00% |
| 2F - 0.05% F | 3.73E+02 | 2.06E+00 | 0.55% |
| 3F - 0.1% F | 2.37E+02 | 4.49E+00 | 1.89% |
| 4F - 1% F | 3.98E+02 | 2.71E+01 | 6.81% |
| 5F - 10% F | 3.35E+02 | 2.31E+02 | 68.87% |
| 6F - 100% F | 6.10E+02 | 7.43E+02 | 121.88% |

TABLE 16
Sample Loss

| Sample | Fetal Loss | Maternal Loss |
|---|---|---|
| 1F | | 58% |
| 2F | 78.75% | 70% |
| 3F | 67.39% | 50% |
| 4F | 67.93% | 74% |
| 5F | 63.02% | 77% |
| 6F | 75.36% | 75% |
| Average Loss | 70.49% | 67% |

We are aiming for a 1000× enrichment. From this experiment, it has been found that column based enrichments are not suitable for rare cell isolation, but for cell populations of at least 10% or higher. Column based methods cause dramatic loss in number of cells, and so an in-solution based technology was used in order to minimize cell loss and processing steps that might stress the cells.

### Example: 6: In-Solution Enrichment

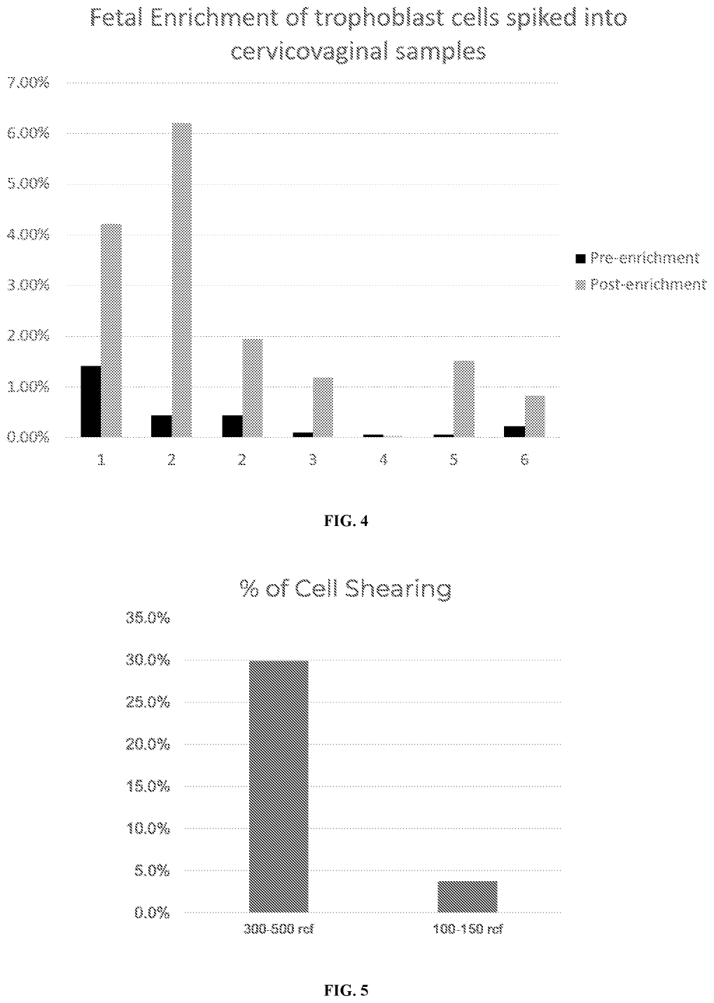

JAR male trophoblast cells were spiked into non-pregnant cervicovaginal cells at a ratio approximately 1/2000 cells. Miltenyi MACS magnetic beads were used with different antibodies bound to a fluorophore APC or PE. Anti-APC or Anti-PE beads were used to bind antibody for magnetic bead separation. Pre and post enrichment aliquots were measured using the RNaseP and Y chromosome qPCR assay to quantify enrichment percentage. Different incubation buffers with changes in salt ions were also tested. Data is shown in FIG. 4 and Table 17.

TABLE 17
Round 1 In-solution enrichment

| | | | | | Pre- | Post- | Fold |
|---|---|---|---|---|---|---|---|
| Round | Antibody | Conditions | Rounds | Beads | enrichment | enrichment | change |
| 1 | HLA-g | Overnight | 1 | MACS PE | 1.41% | 4.22% | 3 |
| | | incubation with | | | | | |
| | | antibody | | | | | |
| 2 | HLA-g | HBSS buffer | 1 | MACS PE | 0.44% | 6.21% | 14 |
| 2 | HLA-g | DPBS buffer | 1 | MACS PE | 0.44% | 1.95% | 4 |
| 3 | HLA-g | IgG antibody based | 1 | MACS IgG | 0.10% | 1.19% | 12 |
| | | mag beads with | | | | | |
| | | overnight incubation | | | | | |
| 4 | HLA-g | IgG antibody with | 1 | MACS IgG | 0.06% | 0.04% | 1 |
| | | no overnight | | | | | |
| | | incubation | | | | | |
| 5 | FOLR1/ | Maternal depletion | 2 | GE Mag | 0.06% | 1.51% | 26 |
| | Trop2 | with second round | | streptavidin | | | |
| | | of fetal depletions | | (m) + M-280 | | | |
| | | | | IgGM (F) | | | |
| 6 | FOLR1 | Maternal depletion | 1 | polystyrene | 0.22% | 0.83% | 4 |
| | | only | | size selection | | | |
| | | | | beads | | | |

HBSS containing calcium and magnesium helps with antibody binding, and a combination of streptavidin and IgG mouse beads for maternal depletions and fetal enrichments, respectively, gives highest enrichment amount.

Five pregnant clinical samples collected in 1:8 Norgen buffer in 0.5×PBS were collected and processed via optimized buffer dissociation protocols. Cells were passed through a 30 uM filter to eliminate large cells, clumps, and duplets. Cells were counted, and then were subjected to 3 rounds of in-solution maternal depletion (CD45, FSHR, LCHGR). CD270 was not used in this round due to limited quantities of the antibody present at the time. Cells were counted again to determine number of cells depleted. The cells were then stained with 3 fetal trophoblast specific antibodies (CD114, FOLR1, and Trop2), and cells that were positive for all three markers were quantified on flow cytometry to determine number of fetal trophoblast cells. Maternal depleted samples were then put on FACS and cells positive for CD114, FOLR1, and Trop2 were isolated. DNA was extracted and fetal fraction was determined for this in solution maternally depleted and FACS fetal enriched samples.

TABLE 18
Round 2 enrichment

| | | | | | | % | | |
|---|---|---|---|---|---|---|---|---|
| | | | | Estimated | % SRY | SRY | | |
| | | | | % fetal | signal | signal | Estimated | |
| | Before | After | % | before | before | after | fold | |
| Sample | Depletion | depletion | depleted | enrichment | FACS | FACS | enrichment | Comments |
| TB112 | 1.17E+07 | 2.61E+06 | 77.69% | 0.05% | 2% | 21% | 420 | |
| TB116 | 4.67E+06 | 9.14E+05 | 80.43% | 0.05% | Not | 100% | 2000 | |
| | | | | | measured | | | |
| TB117 | 9.20E+06 | 3.45E+06 | 62.50% | 0.05% | Not | 14% | 280 | |
| | | | | | measured | | | |
| TB118 | 7.25E+06 | 1.14E+06 | 84.28% | 0.05% | Not | 0% | Unknown | Female |
| | | | | | measured | | | fetus not |
| | | | | | | | | detected |
| | | | | | | | | with SRY |
| | | | | | | | | assay |
| TB119 | 5.95E+06 | 3.07E+05 | 94.84% | 0.05% | Not | 51% | 1020 | |
| | | | | | measured | | | |

A two technology approach works well to achieve a 1000× enrichment. In solution depletion of maternal and FACS enrichment of fetal trophoblast cells. A second in-solution technology for fetal enrichment instead of FACS is being developed by employing to types of magnetic beads. Maternal antibodies are biotin labeled and to large magnetic beads coated with streptavidin. This ensures rapid capture of maternal cells with the ability to capture large numbers of cells. The second round of in solution fetal enrichments, anti-IgG mouse magnetic beads are incubated with anti-human fetal antibodies that are raised in mouse hosts, in order to bind the IgG tag within the antibody. This means that fetal and maternal chemistries of antibody binding are different, so that there is no cross reactivity between the maternal and fetal antibodies. A long incubation of fetal trophoblast cells ensures that the small number of cells in solution are sufficiently bound before magnetic separation.

Eleven clinical samples were processed and split into a large and small cell size aliquot. Samples were submitted to 4 rounds of maternal depletions before being put on flow cytometer to analyze trophoblast positive cells. Each round of depletion was carried out at 4° C. for at least 8 hours. Maternal antibodies were conjugated to GE magnetic streptavidin speed beads using biotin conjugated antibodies. Each round of depletion on use one maternal antibody in the following order: CD45, LHCGR, CD270, then FSHR. Cells were counted before and after depletions, and final depleted samples were stained with anti-Trop2 PE and analyzed on flow cytometer to quantify proportion of Trop2 positive cells.

TABLE 19
Round 3 Enrichment

| | | | | | # | Total |
|---|---|---|---|---|---|---|
| | | | | | Trop2 + | cells |
| | # cells | # cells | % | % | Cells in | per |
| Sample | start | after | depletion | Trop2+ | sample | patient |
| 01L | 1.25E+06 | 1.35E+05 | 89.16% | 0.45% | 608 | 1033 |
| 01S | 4.58E+05 | 3.08E+04 | 93.28% | 1.38% | 425 | |
| 02L | 3.63E+05 | 8.70E+04 | 76.03% | 2.49% | 2166 | 2993 |
| 02S | 5.39E+05 | 1.45E+05 | 73.10% | 0.57% | 827 | |
| 03L | 3.02E+06 | 5.35E+05 | 82.26% | 0.24% | 1284 | 1921 |
| 03S | 7.48E+05 | 1.30E+05 | 82.62% | 0.49% | 637 | |
| 1L | 8.26E+05 | 2.74E+05 | 66.83% | 0.15% | 411 | 1401 |
| 1S | 1.75E+06 | 4.50E+05 | 74.29% | 0.22% | 990 | |
| 2L | 4.22E+05 | 1.00E+05 | 76.30% | 0.33% | 330 | 1177 |
| 2S | 1.13E+06 | 1.97E+05 | 82.61% | 0.43% | 847 | |
| 3L | 1.91E+05 | 3.35E+04 | 82.41% | 1.38% | 462 | 1326 |
| 3S | 3.14E+06 | 1.20E+05 | 96.18% | 0.72% | 864 | |
| 4L | 3.20E+04 | 8.43E+03 | 73.63% | 1.04% | 88 | 640 |
| 4S | 1.09E+06 | 6.74E+04 | 93.83% | 0.82% | 553 | |
| 5L | 7.46E+05 | 1.59E+05 | 78.69% | 1.66% | 2639 | 9900 |
| 5S | 2.09E+06 | 2.65E+05 | 87.29% | 2.74% | 7261 | |
| 6L | 3.33E+06 | 5.52E+05 | 83.41% | 0.75% | 4140 | 14745 |
| 6S | 5.17E+06 | 1.05E+06 | 79.69% | 1.01% | 10605 | |
| 7L | 4.63E+05 | 4.43E+04 | 90.43% | 0.86% | 381 | 1001 |
| 7S | 1.94E+06 | 4.43E+05 | 77.13% | 0.14% | 620 | |
| 8L | 9.87E+05 | 2.23E+05 | 77.41% | 0.36% | 803 | 899 |
| 8S | 2.32E+05 | 1.58E+04 | 93.20% | 0.61% | 96 | |

This data shows consistent and reproducible depletion of maternal material by 85%+/−5%. Average fetal content after depletion is on average 0.86%, which is suitable for high purity FACS sorting of cells.

Eight samples from the same patient were subjected to alternate rounds of enrichment/depletion using designated antibodies. Maternal antibodies were conjugated to GE-magnetic streptavidin speed beads using biotin, and fetal antibodies conjugated to Thermo M280 mouse IgG magnetic dynabeads using mouse anti-human fetal antibodies. Antibody bound beads were incubated with cells for at least 8 hours for each round. Four samples were incubated using DPBS with 0.5% BSA/EDTA, and the other 4 were incubated with the TCA Norgen buffer with 0.5% BSA/EDTA. Maternal antibodies used were CD45, LHCGR, CD270, and FSHR. Fetal antibodies were Trop2, HLA-g, SLC40A1, and FOLR1.

TABLE 20
Round 4 Enrichment

| | Rounds | Rounds | | # cells | | |
|---|---|---|---|---|---|---|
| | maternal | fetal | # cells | after | % | |
| Sample | depletion | enrichment | starting | depletion | depleted | Order of depletion/enrichment |
| D1 | 4 | 2 | 1.00E+06 | 6.62E+03 | 99.34% | CD45, LHCGR, CD270, FSHR, |
| | | | | | | Trop2 |
| D2 | 4 | 2 | 1.00E+06 | 8.95E+03 | 99.11% | CD45, LHCGR, CD270, FSHR, |
| | | | | | | (Trop2, HLA-g, SLC40A1, |
| | | | | | | FOLR1), (Trop2, HLA-g, |
| | | | | | | SLC40A1, FOLR1) |
| D3 | 2 | 2 | 1.00E+06 | 6.73E+03 | 99.33% | Trop2, Trop2, (CD45, LHCGR, |
| | | | | | | CD270), (CD45, LHCGR, |
| | | | | | | CD270) |
| D4 | 2 | 2 | 1.00E+06 | 7.57E+03 | 99.24% | (Trop2, HLA-g, SLC40A1, |
| | | | | | | FOLR1), (Trop2, HLA-g, |
| | | | | | | SLC40A1, FOLR1), (CD45, |
| | | | | | | LHCGR, CD270, FSHR), (CD45, |
| | | | | | | LHCGR, CD270, FSHR) |
| T1 | 4 | 2 | 1.00E+06 | 1.11E+04 | 98.89% | CD45, LHCGR, CD270, FSHR, |
| | | | | | | Trop2 |
| T2 | 4 | 2 | 1.00E+06 | 7.07E+03 | 99.29% | CD45, LHCGR, CD270, FSHR, |
| | | | | | | (Trop2, HLA-g, SLC40A1, |
| | | | | | | FOLR1), (Trop2, HLA-g, |
| | | | | | | SLC40A1, FOLR1) |
| T3 | 2 | 2 | 1.00E+06 | 4.06E+03 | 99.59% | Trop2, Trop2, (CD45, LHCGR, |
| | | | | | | CD270), (CD45, LHCGR, |
| | | | | | | CD270) |
| T4 | 2 | 2 | 1.00E+06 | 1.14E+04 | 98.86% | (Trop2, HLA-g, SLC40A1, |
| | | | | | | FOLR1), (Trop2, HLA-g, |
| | | | | | | SLC40A1, FOLR1), (CD45, |
| | | | | | | LHCGR, CD270, FSHR), (CD45, |
| | | | | | | LHCGR, CD270, FSHR) |

Fetal enrichments first were able to reduce the rounds of enrichment from 6 to 4 with no difference in overall reduction of number of cells. Consistent 98-99% depletion of overall cell content was achieved.

### Example 7: Sample Processing

After 6 days in buffer, cell membranes lose their integrity and may be more prone to degradation in sample processing procedures, so protocols for sample processing were optimized. For example, reducing the speed of centrifugations, reducing the number of centrifugations, utilizing technologies that minimize cell processing steps (i.e., switch from column based bead selection to in solution based methods). Cells were stained with appropriate antibodies and subjected to either centrifugation of 300-500 RCF or 100-150 RCF to determine how centrifugation speeds impact ability to keep cells intact. Cells were put on flow cytometer and the percentage of intact cells was measured in both the unprocessed and processed cell fractions to determine change in percentage of intact cells within the sample. The absolute number of intact cells is not important, as it will vary from sample to sample, however the change in intact cells is important to determine optimal processing conditions.

TABLE 21
Sample Processing Effects

| | | | % Change in intact due to |
|---|---|---|---|
| Centrifugation | % Intact | % Intact | degradation of cells |
| Speeds (RCF) | Unprocessed | Processed | in processing |
| 300-500 | 37.8% | 7.9% | −79.1% |
| 100-150 | 11.1% | 7.3% | −34.0% |

Centrifugation speed dramatically affects cells staying intact during processing. This is likely due to the fact that even though higher rotational forces cause shearing of cell membranes. FIG. 5 shows a comparison of the percentage of cells sheared of total cells in samples processed at 300-500 rcf compared to samples processed at centrifugation speeds between 100-150 rcf.

### Example 8: Preservation of Cells in Cervicovaginal Samples

To examine the physical characteristics of cells in cervicovaginal samples, experiments were conducted to ascertain cell scatter on flow cytometry, and the ratio of dead cells present within the sample.

Cervicovaginal samples from 3 pregnant and 3 non-pregnant women (through WIRB approved protocols) was collected using the tampon-based collection system and preservation device as described in Publication No. US2016-0324506 and US-2019-0125316 (referred to herein as “tampon-based collection and preservation device”). The pregnant samples were pooled together, and separately, the non-pregnant samples were pooled together.

The device contained Whole cell preservation (WCPS) buffer (Norgen cfDNA/cfRNA buffer diluted 1:8 in 0.5×PBS) in the breakable pouch, such that upon closure of the device containing the tampon with the collected cervicovaginal sample, the buffer was released and contacted the sample. After arrival at the laboratory, the cells were dissociated from the tampon, and filtered through a 30 uM filter to remove any cell clumps. The cells were then stained with propidium iodide (passive transport dye that stains dead cells) as follows: cells were stained in cold 1×PBS with a PI concentration of 0.5%. Cells were incubated in stain for 10 minutes, centrifuged for 5 minutes at 100 rcf and washed with 1×PBS. Sample were then resuspended in MACS running buffer before running on flow cytometer.

Cells were put onto flow cytometer and examined on side scatter (granularity) and forward scatter (size).

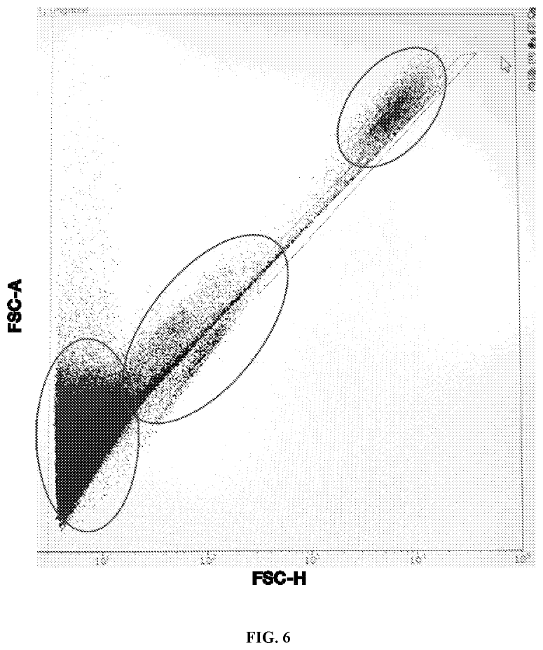

FIG. 6 shows the forward scatter pattern for an exemplary sample. The forward scatter area by forward scatter height provides a relative indication of the size of individual cells and quantification of doublet events. The diagonal in the graph presented shows the aggregation of single cells (the Y access shows minimal aggregation of cell doublets). Three main meta-population were represented within cervicovaginal samples. Population I) represents the main human cell populations, which is generally be larger in size than prokaryotic cells. Population II) represents bacterial and fungal cell populations in the 2-5 uM range. Population III) represents debris and anything else in the sample under 2 uM in size.

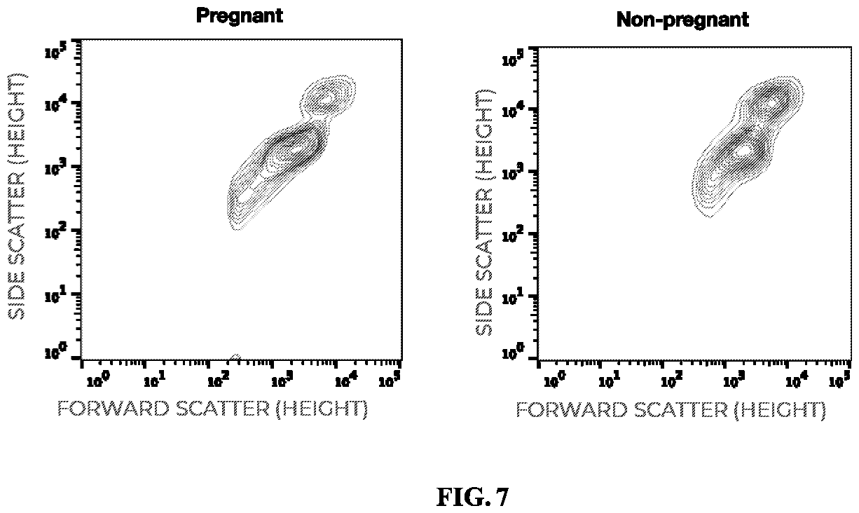

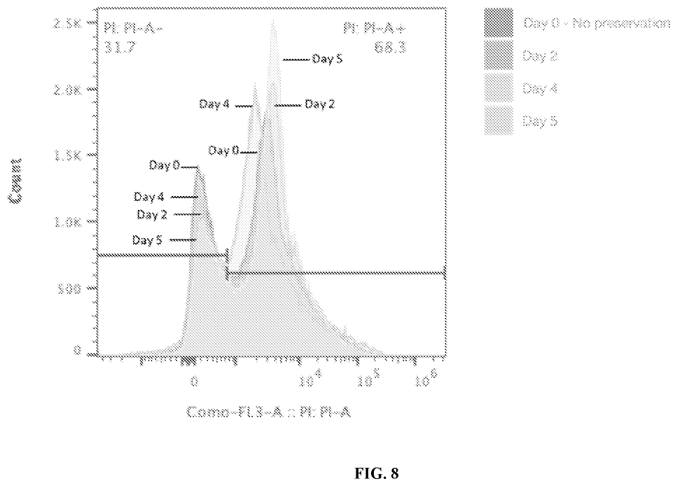

The flow cytometer was gated on Population I to examine the size and granularity of the human cell populations between pregnant and non-pregnant controls. The samples are shown in FIG. 7. Generally each group produced morphologically similar cells populations, a very tightly clustered granular population, and more dispersed population below that varies in cell size and granularity.

### Example 9: Preservation of Cells

Similar to Example 8, samples were collected using WCPS buffer-tampon-based collection and preservation device. Samples were removed from the collection device using the harvester to compress tampon and elute cells into solution. Day zero was eluted with 1×PBS instead of WCPS buffer to assess the native state of the cells at time of collection without the influence of preservation buffer on the sample. Samples preserved in the device from days 2, 4 and 5 were eluted in WCPS buffer with the harvester tool after designated days of incubation. All samples were then stained with propidium iodide (see Example 8). For flow cytometry, cells were gated on intact cells, gating out small bacteria and debris from analysis. Intact cells were visualized on PI emission spectrum (535 nM)—X-axis, and total event counts on Y-axis.

As shown in FIG. 8, the samples between 0-5 days of preservation gave a similar ratio of dead to live cells and demonstrated the stability provided by the collection and preservation system. Each color represents a single tampon sample from a single pregnant subject.

### Example 10: Enrichment by Density Gradient

Cervicovaginal samples obtained using the WCPS tampon-based collection and preservation device were processed into a single cell suspension. After elution of the cells as described in Example 8, the eluted cells were centrifuged at 100 rcf/g for 10 minutes to pellet the cells. The cell pellet was resuspended in 800 ul of 1×PBS with EDTA and 0.5% BSA and 100 ul of Collagenase IV and 25 ul of DNase I was added to each sample, and the sample was then gently mixed. The samples were incubated for 30 min at 37° C. with gentle mixing at 550 rpm. Following incubation, the cells were pelleted at 100 g for 5 minutes. The pellet was gently resuspended, overlayed onto BSA, and centrifuged at 800 rpm for 15 minutes. The supernatant was removed and the pellet was resuspended in wash buffer and placed on ice. The sample was then filtered through a 30 uM filter, with repeated washed (5 total) to remove clumped and doublet cells and provide a single cell suspension in buffer for application to the density gradient.

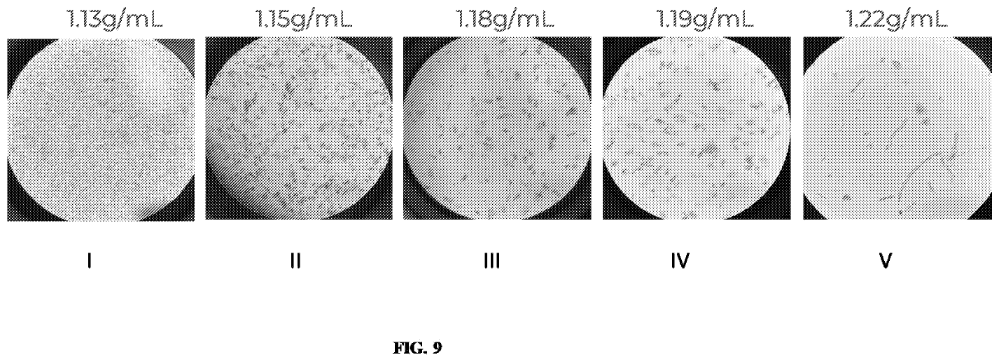

Discontinuous density gradients were created using OptiPrep, 60% iodixanol mixture in water with a density of 1.32 g/mL. 13 gradations of densities were created from 1.11 g/mL to 1.22 g/mL. Cell suspension were layered on top of density gradients and spun in a bucket rotor centrifuge at 3,400 rcf for 15 minutes. Each layer was removed and visualized under a microscope to identify morphological character of cell layer and quantification of debris. A visualization for exemplary layers is shown in FIG. 9.

Five main layers of cellular material separated throughout the density gradients. Layer I contained mostly bacteria and small debris particles, with a few very large disc shaped cells whose surface area may have impaired proper gradation. Layers II and III contained many intact cells and larger pieces of debris scattered throughout. Layer IV contained a pure population of intact cells with no visible debris present and Layer V contained filamentous particles, likely cotton fibers from the tampon. Layer IV presented an ideal population due to its population of cells and lack of debris that could cause autofluorescence.

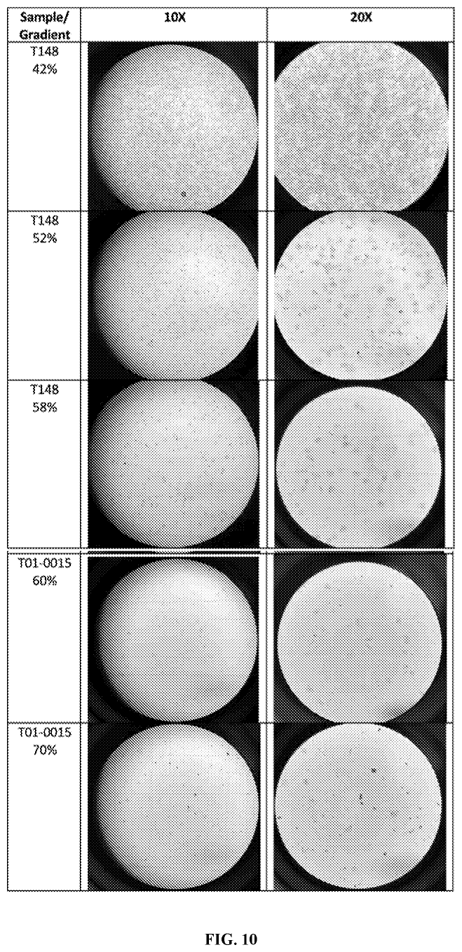

A second density gradient experiment was performed using cervicovaginal samples from two tampon samples from one subject pooled together. From layers were separately collected from points in the gradient at 42%, 52%, 58%, 60%, 70% and visualized by microscopy as shown in FIG. 10. Fetal fraction of each layer was assayed by a Y chromosome qPCR assay. Briefly, 1 ng of DNA was pre-amplified with gene specific primers to target genes using Takara's Pre-Amp mix and 10 cycles of PCR. PCR product was then diluted 1:50, and 3 ul were run using Taqman primer probe technology and amplified for 40 cycles. All samples were normalized to GAPDH and a set of known standards with precise ratios of Y chromosome DNA from 0.05% to 20% Y chromosome content. Fetal fraction was then calculated based on the standard curve. Primers are shown in Table 22.

TABLE 22
Y chromosome gene specific primer/probes

| | | Product |
|---|---|---|
| Primer | Sequence | Length |
| TSPY.71 | GGGCCAATGTTGTATCCTTCTC | 84 |
| Forward | | |
| TSPY.71 Reverse | GCCCATCGGTCACTTACACTTC | |
| TSPY.71 Probe | TCTAGTGGAGAGGTGCTC | |
| TSPY.76 | GCTATTGAGTTGTTGGGAGTTCCTT | 137 |
| Forward | | |
| TSPY.76 Reverse | AAGTGCTTCACCACACCATGAAAA | |
| TSPY.76 Probe | CTGTGACTATTCCC | |

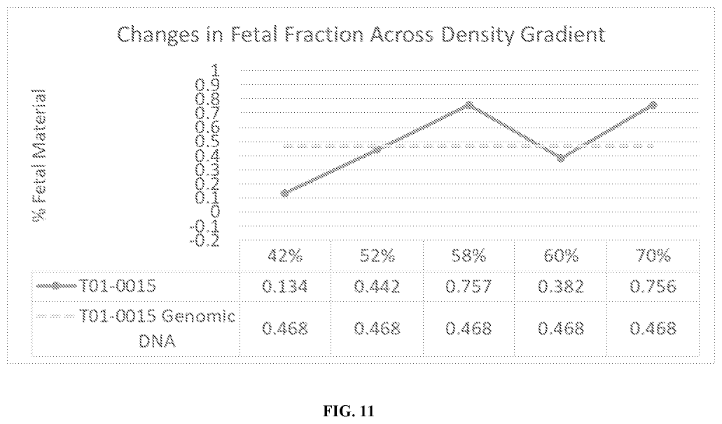

Fetal fraction of each layer is shown in FIG. 11. The doted blue line represents the native baseline ratio of fetal Y chromosome DNA in the pooled sample. This is at 0.468%. The solid blue line represents the percent enrichment in each of the gradient layers after centrifugation. The difference between the baseline ratio and the enrichment ratio represents the percent enrichment due to the gradient isolation. Y axis represents percent Y chromosome and X axis represents each aliquot of cells isolated in the density gradient (from 42%-70%). The graph shows that 58% and 70% gradient layers show an increase in baseline fetal fraction from 0.468% to 0.76%. The 70% gradient layer may have an enrichment of free-floating fetal nuclei.

### Example 11: Enrichment Using Nuclear Isolation

Nuclei isolated from the preserved cervicovaginal samples offers benefits of specific nuclear membrane targets, elimination of debris through lysis, reduction in autofluorescence and non-specific binding of sticky or degraded cell membranes. To test if nuclei isolation would provide specific cell-type enrichment, the following experiment was performed.

Free floating nuclei were isolated using a BSA cushion, where 800 ul of 10% BSA was added to a microtube, and a cell suspension in 1×PBS was gently added on top of the BSA cushion. Cells were centrifuged at 800 rpm for 15 minute's. The top layer was then resuspended in MACS wash buffer and stained with anti-ERVFR1-FITC at 1 mg antibody per 1×10⁶ cells. FACS sorting was performed on the stained samples and cells were gated on ERVFR1 positivity, and autofluorescent cells were excluded by gating out positive events on the empty 405 laser channel. Population of nuclei were then back gated onto unstained control sample to visualize distinct population of positive nuclei. This population of cells was then sorted into a microtube for DNA extraction

From the isolated nucleic, DNA was extracted and assayed for Y chromosome as outlined in Example 10. Y chromosome was present at 1.4% of the enriched, cell sorted sample, whereas the baseline, pre-enriched fraction had a Y chromosome ratio of 0.00010% which represents a 1400 fold enrichment from the starting sample.

### Example 12: Cell Population Analysis

187 cervicovaginal tampon samples from 55 participants were collected using tampon-based collection and preservation device with Nucleic acid preservation buffer (Norgen Swab Buffer diluted 2:1 in 1×PBS). RNA was extracted from total cells lysate using Ambion miRVana kits to capture total RNA. For all samples, RNA sequencing libraries were prepped (using standard Illumina protocols), and then human RNA was enriched using IDTs exome panel (coding plus regions of human genome), to generate reads only for human material (TARGETED). Libraries were sequence on a NextSeq500 using 75 bp paired end reads. For Targeted sequences, Fastq files were processed, trimmed and aligned to the human genome, and transcripts were quantified. Normalized read counts were then imported into xCell (xcell.ucsf.edu), a database of 67 individual cell types and their corresponding gene signatures, to quantify the abundance rank of all cell types in cervicovaginal samples. For all 187 samples, the top abundant cell types were visualized.

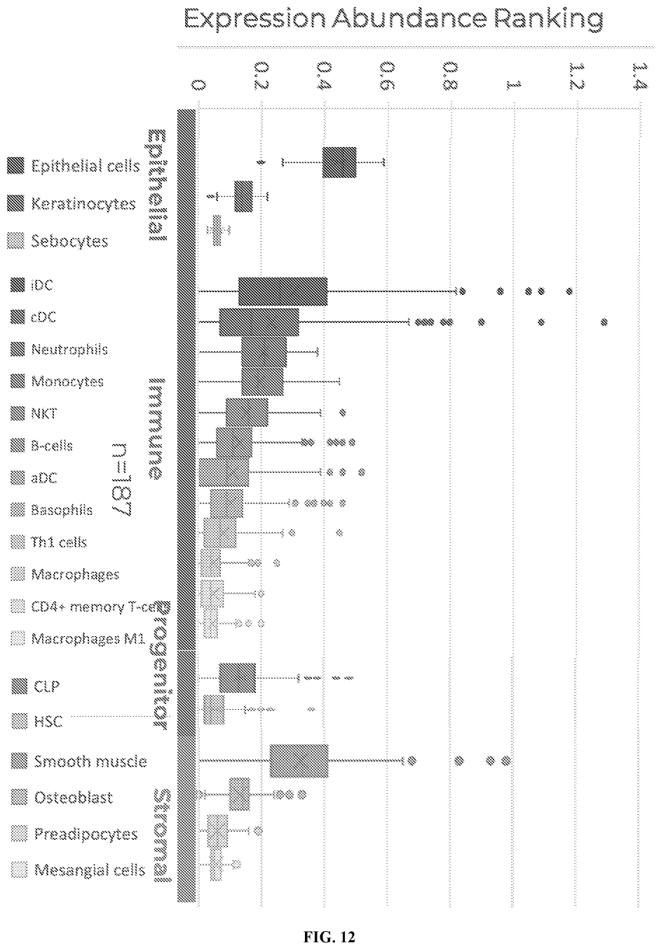

FIG. 12 shows that RNA expression and cell type analysis reveal that cervicovaginal samples contain diverse populations of immune cells, epithelial, stromal, and stem cells. Dendritic cells, regular epithelial cells and smooth muscle endothelial cells represent that most abundant cell populations. This represents the basic cell composition of cervicovaginal samples collected using the tampon-based sampling methodologies described herein.

### Example 13: Reducing Autofluorescence

Cervicovaginal samples were collected from pregnant women (5 samples from 3 participants) and treated as described in Example 8 for PI staining.

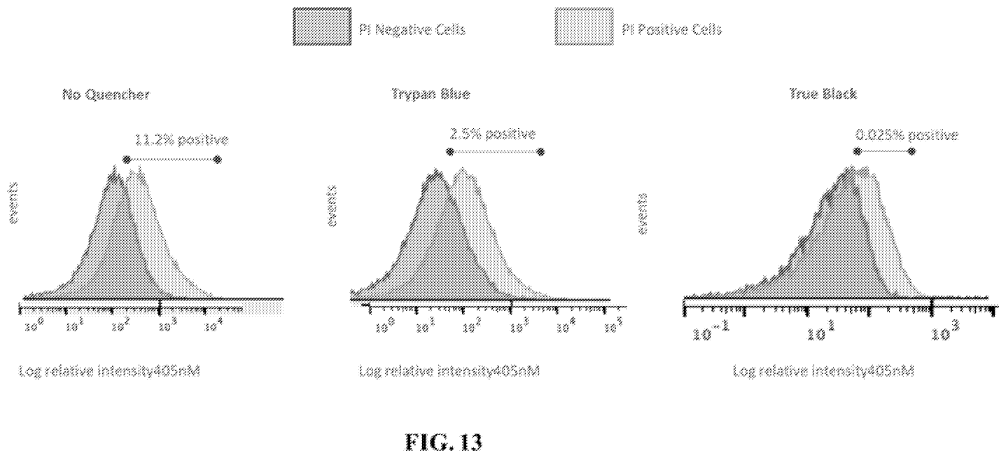

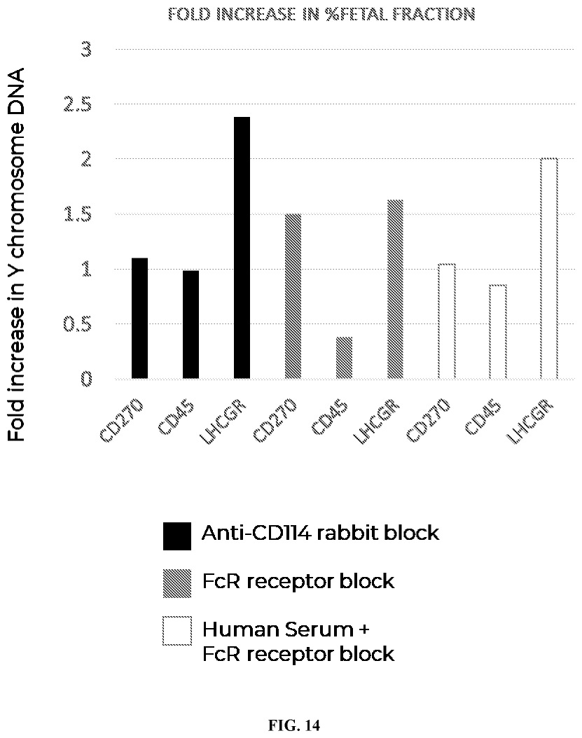

Samples were separated into three experimental conditions: two different quencher solutions (Trypan Blue and True Black) at 0.05% concentration and no quencher. Results were analyzed on the SONY SH800 FACS sorter. Cells were put onto FACS machine and examined on side scatter (granularity), forward scatter (size), 405 laser (autofluorescence) and 488 laser (PI). FIG. 13 shows representative reduction in autofluorescence per condition for one of the 5 samples analyzed. Autofluorescence among dead, PI positive cells was dramatically reduced with True Black (Biotium) from 11.2% to 0.025%.

### Example 14: Enrichment Using Maternal Depletion

Different blocking solutions were investigated to improve binding of specific maternal antibodies for maternal depletion of cells. 6 tampon samples were collected in WCPS buffer-tampon-based collection and preservation device from 2 pregnant participants and samples were pooled together. Cells were dissociated and filtered as described in Example 8, and samples were aliquoted into 12 separate aliquots. 4 aliquots were reserved for each experimental condition. The following blocking strategies were utilized: No blocking, FcR fragments blocking, Human Serum+FcR fragments, and CD114 Rabbit (a non-mouse, non-target antibody used to saturate non-specific binding sites that was raised in a different host than the target antibodies). Samples were incubated with respective blocking agent for 1 hour at 4° C., and then either CD270, CD45, or LHCGR antibody-conjugated magnetic beads were added to the tubes to bind maternal cells. The bead and sample mixtures were incubated on a rotor at 4° C., for at least 4 hours. Samples were put on a magnet, and both negative and positive populations were collected. DNA was extracted from each bead population and Y chromosome content was measured using the Y chromosome qPCR assay as described in Example 10.

The change in percent fetal fraction was calculated between each paired positive and negative fraction for each antibody and blocking reagent. The fold increase was calculated by comparing each blocking condition to the no-blocking control samples. Fold increase in percent fetal fraction is plotted in FIG. 14. The results indicate that the enrichment-potential for different antibodies perform optimally in different blocking buffers, indicating that a step-wise enrichment strategy with multiple blocking and enrichment rounds would provide additional depletion of maternal material. For example, for LHCGR, both human serum+FCR or a non-specific antibody (CD114) allow for greater fetal enrichment. CD270 performs optimally with an FCR only blocking strategy. Enrichment with CD45 antibody was not significantly increased with blocking, however, because FcR receptors are found on immune cell populations that are targeted by CD45, blocking with FCR generally decreased the enrichment of the fetal fraction.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A composition comprising: (a) a population of trophoblast and trophoblast-derived cells; (b) a stabilization buffer, wherein the stabilization buffer comprises a preservative; and (c) a cellulosic collection device, wherein at least a subset of the population of cells are intact and inviable.

2. The composition of claim 1, wherein the population of trophoblast and trophoblast-derived cells comprises one or more of a Hofbauer cell, a cytotrophoblast, a syncytiotrophoblast, an extravillous trophoblast, a villous core stroma, or a fetal vascular cell.

3. The composition of claim 1, wherein the cellulosic collection device comprises a tampon, a pad, a plug, or a swab.

4. The composition of any of claim 1, wherein at least a portion of the population of cells are associated with the cellulosic collection device.

5. The composition of claim 1, wherein at least 50%, at least 60%, or at least 70% of the population of cells are intact after 1, 2, 3, 4, 5, 6, 7, 8, 9, or 10 days in the stabilization buffer at room temperature.

6. The composition of claim 1, wherein the stabilization buffer maintains the size of the cells in the population within a range of 0.5 to 1× as compared with the size of the cells prior to contact with the stabilization buffer.

7. The composition of claim 1, wherein the population of cells comprises at least 100 cells or at least 500 cells.

8. A composition comprising: (a) a population of trophoblast and trophoblast-derived cells; and (b) a stabilization buffer, wherein the stabilization buffer comprises a preservative and wherein at least a subset of the population of cells are inviable and at least 50%, at least 60%, or at least 70% of the population of cells are intact after 1, 2, 3, 4, 5, 6, 7, 8, 9, or 10 days in the stabilization buffer at room temperature

9. The composition of claim 8, wherein the population of trophoblast and trophoblast-derived cells comprises one or more of a Hofbauer cell, a cytotrophoblast, a syncytiotrophoblast, an extravillous trophoblast, a villous core stroma, or a fetal vascular cell.

10. The composition of claim 8, wherein the stabilization buffer maintains the size of the cells in the population within a range of 0.5 to 1× as compared with the size of the cells prior to contact with the stabilization buffer.

11. The composition of claim 8, wherein the stabilization buffer maintains the size of the cells in the population within a range of ±20%, ±10%, or ±5% as compared with the size of the cells prior to contact with the stabilization buffer.

12. The composition of claim 8, further comprising a cellulosic collection device.

13. A composition comprising: (a) an enriched population of intact and nonviable trophoblast and trophoblast-derived cells, wherein the population of cells comprises between 0.5% to 50% trophoblast and trophoblast-derived cells and at least 50%, at least 60%, or at least 70% of the enriched population are intact cells.

14. The composition of claim 13, wherein the population of cells comprises at least 0.5%, 1%, 2%, 5%, 10%, 20%, 25%, 30%, 40% or 50% trophoblast and trophoblast-derived cells.

15. The composition of claim 13, wherein the population of cells is enriched for trophoblast and trophoblast-derived cells between about 2-fold and 2000-fold.

16. The composition of claim 13 wherein the population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold.

17. The composition of claim 13, wherein the population of trophoblast and trophoblast-derived cells comprises one or more of a Hofbauer cell, a cytotrophoblast, a syncytiotrophoblast, an extravillous trophoblast, a villous core stroma, or a fetal vascular cell.

18. The composition of claim 17, wherein the enriched population of cells comprises at least two cell types selected from the group consisting of a cytotrophoblast, a syncytiotrophoblast, and extravillous trophoblast.

19. The composition of claim 17, wherein the enriched population of cells comprises at least cytotrophoblasts or syncytiotrophoblasts.

20. The composition of claim 13, further comprising a binding agent that specifically binds to a cell surface marker.

21. A method of obtaining a stabilized population of cells, the method comprising: (a) placing a cellulosic collection device in or near a vagina of a pregnant subject to collect vaginal fluid comprising a plurality of cells comprising trophoblast and trophoblast-derived cells; and (b) contacting the cellulosic collection device comprising the plurality of cells with a stabilization buffer, wherein at least a subset of the plurality of cells are intact and inviable.

22. The method of claim 21, wherein the plurality of trophoblast and trophoblast-derived cells comprises one or more of a Hofbauer cell, a cytotrophoblast, a syncytiotrophoblast, an extravillous trophoblast, a villous core stroma, or a fetal vascular cell.

23. The method of claim 21, wherein at least 50%, at least 60%, or at least 70% of the plurality of cells are intact after 1, 2, 3, 4, 5, 6, 7, 8, 9, or 10 days in the stabilization buffer at room temperature.

24. The method of claim 21, wherein the stabilization buffer maintains the size of the cells in the plurality within a range of 0.5 to 1× as compared with the size of the cells prior to contact with the stabilization buffer.

25. The method of claim 24, wherein the stabilization buffer maintains the size of the cells in the plurality within a range of ±20%, ±10%, or ±5% as compared with the size of the cells prior to contact with the stabilization buffer.

26. The method of claim 21, wherein the plurality of cells comprises at least two of a Hofbauer cell, a cytotrophoblast, a syncytiotrophoblast, an extravillous trophoblast, a villous core stroma, or a fetal vascular cell.

27. The composition of claim 21, wherein the plurality of cells comprises at least one of a cytotrophoblast or a syncytiotrophoblast.

28. The method of claim 21, wherein the cellulosic collection device is in or near the vagina for at least 10, 20, 30, 40, 50, 60, 90, 120, 150, 180, 240, 300, 360, 420, or 480 minutes.

29. The method of claim 21, further comprising eluting the plurality of cells or a portion thereof from the cellulosic collection device to obtain an isolated stabilized population of cells.

30. The method of claim 29, further comprising subjecting the isolated stabilized population of cells to one or more enrichment steps to obtain an enriched population of cells.

31. The method of claim 30, wherein the enriched population of cells is enriched in trophoblast cells, trophoblast-derived cells, or trophoblast and trophoblast-derived cells.

32. The method of claim 30, wherein the enriched population of cells is enriched in one or more of Hofbauer cells, cytotrophoblasts, syncytiotrophoblasts, extravillous trophoblasts, villous core stroma cells, or fetal vascular cells.

33. The method of claim 30, wherein the enriched population of cells comprises at least 0.5%, 1%, 2%, 5%, 10%, 20%, 25%, 30%, 40% or 50% trophoblast and trophoblast-derived cells.

34. The method of claim 30, wherein the enriched population of cells is enriched for trophoblast and trophoblast-derived cells between about 2-fold and 2000-fold.

35. The method of claim 30, wherein the enriched population of cells is enriched for trophoblast and trophoblast-derived cells by at least about 2-fold, 5-fold, 10-fold, 20-fold, 50-fold, 100-fold, 500-fold, 1000-fold, 1400-fold, 1500-fold, or 2000-fold.

36. The method of claim 30, wherein the enriched population of cells is depleted of maternal cells.

37. The method of claim 36, wherein the enriched population of cells is depleted of maternal cells between about 80% to about 99.9% as compared to the isolated stabilized population of cells.

38. The method of claim 36, wherein the enriched population of cells comprises between about 50% to about 99.5% maternal cells.

39. The method of claim 30, wherein the enrichment step comprises contacting with a binding agent that specifically binds to a cell surface marker, flow cytometry, density gradient centrifugation, and nuclei isolation.

40. The method of claim 30, comprising 2, 3 or 4 enrichment steps.

41. The method of claim 40, wherein the enrichment step comprises contacting with a binding agent that specifically binds to a cell surface marker and separating at least a subset of the cells bound by the one or more binding agents to thereby form the enriched population of cells.

42. The method of claim 41, wherein the cell surface marker is a fetal cell surface marker.

43. The method of claim 41, wherein the cell surface marker is a maternal cell surface marker.

44. The method of claim 41, wherein the cell surface marker comprises FSHR, CD45, LHCGR, CD270, Trop2, FOLR1, CD114 (CSF3R) or SLC40A1.

45. The method of claim 41, wherein the binding agent is selected from the group consisting of an antibody, antibody fragment, a magnetic bead and a detectable label.

46. The method of claim 41, wherein the cell surface marker is a maternal cell surface marker and the enriched population is depleted of maternal cells.

47. The method of claim 41, wherein the cell surface marker is a fetal cell surface marker and the enriched population is enriched in placenta derived fetal cells.

48. The method of claim 41, wherein the cell surface marker is a fetal cell surface marker and the enriched population is enriched in fetal trophoblast cells.

49. The method of claim 41, wherein comprising quantifying a fetal fraction of the plurality of cells.

50. The method of claim 49, wherein quantifying a fetal fraction comprises intracellular staining, RNA analysis, short tandem repeat (STR) analysis, single nucleotide polymorphism (SNP) analysis, Y-chromosome analysis, or combinations thereof.

51. The method of claim 21, wherein the cellulosic collection device is a tampon, a pad, a plug, or a swab.

52. The method of claim 21, further comprising removing mucus from the plurality of cells.

53. The method of claim 21, further comprising analyzing at least one of a nucleic acid, a protein, a carbohydrate, or a lipid from the enriched population of cells.

54. The method of claim 53, further comprising recommending a treatment to the pregnant subject.

55. The method of claim 21, further comprising analyzing at least one of a nucleic acid, wherein the feature comprises a copy number variant, a single nucleotide variant, an insertion, a deletion, an epigenetic modification, a post-translational protein modification, or a combination thereof.

56. The method of claim 55, wherein the epigenetic modification comprises methylation, phosphorylation, ubiquitination, sumoylation, acetylation, ribosylation, citrullination, or a combination thereof.

57. The method of claim 55, further comprising labeling at least one of the nucleic acids prior to analysis.

58. The method of claim 57, wherein the analysis comprises one or more of PCR, sequencing, karyotyping, in situ hybridization, immunofluorescence, FACS, or mass spectroscopy.


SEQUENCE LISTING (6 sequences)

SEQ ID NO: 1 - DNA, 22 bp, Artificial Sequence
  Description of Artificial Sequence Synthetic primer
gggccaatgttgtatccttctc

SEQ ID NO: 2 - DNA, 22 bp, Artificial Sequence
  Description of Artificial Sequence Synthetic primer
gcccatcggtcacttacacttc

SEQ ID NO: 3 - DNA, 18 bp, Artificial Sequence
  Description of Artificial Sequence Synthetic probe
tctagtggagaggtgctc

SEQ ID NO: 4 - DNA, 25 bp, Artificial Sequence
  Description of Artificial Sequence Synthetic primer
gctattgagttgttgggagttcctt

SEQ ID NO: 5 - DNA, 24 bp, Artificial Sequence
  Description of Artificial Sequence Synthetic primer
aagtgcttcaccacaccatgaaaa

SEQ ID NO: 6 - DNA, 14 bp, Artificial Sequence
  Description of Artificial Sequence Synthetic probe
ctgtgactattccc